(19) United States
(12) Patent Application Publication
(10) Pub. No.: US 2026/0102247 A1
(43) Pub. Date: Apr. 16, 2026

(54) HANDLE FOR AN IMPLANT DELIVERY APPARATUS

(71) Applicant: Edwards Lifesciences Corporation, Irvine, CA (US)

(72) Inventors: Grant Jason Gangeness, Carlsbad, CA (US); James William Ventress, Lake Havasu City, AZ (US); Timothy Lee Humphrey, Anaheim, CA (US); Tri D. Tran, Fountain Valley, CA (US); Yuanlong Du, Irvine, CA (US); Andrew Gordon Method, Park City, UT (US); Thomas James Deloss Thompson, Newport Beach, CA (US)

(21) Appl. No.: 19/418,691

(22) Filed: Dec. 12, 2025

Related U.S. Application Data
(63) Continuation of application No. 18/911,013, filed on Oct. 9, 2024.
Application No. 18/911,013 is a continuation of application No. PCT/US2023/018813, filed on Apr. 17, 2023.
(60) Provisional application No. 63/363,162, filed on Apr. 18, 2022.

(51) Int. Cl. A61F 2/24

(52) U.S. Cl. CPC A61F 2/2427

(57) ABSTRACT

A handle for a prosthetic implant delivery apparatus includes a plurality of knobs configured to control corresponding mechanisms of the delivery apparatus. Each knob has a color that is visually distinct from other knobs to indicate a unique function. In some instances, each knob is operably coupled to an indicator viewable through a window of the handle, and the indicator shares the same color as the corresponding knob to convey a functional relationship. Rotation or manipulation of a knob causes a corresponding adjustment mechanism, such as a pull wire, to actuate a feature of the delivery apparatus, for example, to control flex in different regions of a delivery shaft. A method for delivering a prosthetic implant includes manipulating one or more colored knobs to adjust aspects of the delivery apparatus and observing movement of matching-colored indicators to verify operation prior to or during implant deployment.

### CROSS-REFERENCE TO RELATED APPLICATIONS

This application is a continuation of U.S. patent application Ser. No. 18/911,013, filed on Oct. 9, 2024, which is a continuation of International Patent Application No. PCT/US2023/018813, filed on Apr. 17, 2023, which claims the benefit of U.S. Provisional Ser. No. 63/363,162 , filed Apr. 18, 2022. Each related application is incorporated by reference herein.

### FIELD

The present disclosure relates handles for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A delivery apparatus to deliver a prosthetic medical device, such as the prosthetic heart valve delivery apparatus described above, can include an elongated shaft that is inserted into the patient's vasculature. The delivery apparatus can also include a handle that remains outside the patient and can be used to manipulate the shaft.

### SUMMARY

Described herein are prosthetic heart valves, delivery apparatuses, and methods for implanting prosthetic heart valves. The disclosed delivery apparatuses can, for example, be configured to transmit torque exerted on a handle of the delivery apparatus to a distal end of a shaft extending distally from the delivery apparatus via a spine assembly. In some examples, the handle can be used to manipulate the distal end of the shaft and the delivery apparatus can include an indicator assembly (e.g., within the handle, viewable through a window of the handle, etc.) to indicate manipulation of the shaft. As such, the devices and methods disclosed herein can, among other things, overcome one or more of the deficiencies of typical delivery apparatuses.

In one representative example, a handle for a delivery apparatus comprises a housing having an outer wall; at least one window coupled to the outer wall of the housing, the at least one window defining at least one viewing region through the outer wall; and at least one indicator positioned within the housing adjacent to the window, the at least one indicator including a background member and a slider, wherein the slider is configured to slide relative to the background member.

In another representative example, a delivery apparatus comprises a handle including a body and a nose cone positioned distal to the body, wherein the nose cone includes at least one axial runner extending from an inner surface of the nose cone; a spine positioned within the body of the handle, the spine including a lumen; a shaft positioned within the lumen of the spine, the shaft extending distally from the handle; and a spine extension positioned within the nose cone and coupled to the spine, the spine extension at least partially surrounding the shaft, the spine extension including radial projections, wherein each of the radial projections includes one or more mating features defining at least one slot corresponding to the at least one axial runner of the nose cone.

In another representative example, a delivery apparatus comprises a handle; a shaft having a distal end and a proximal end, the shaft extending distally from the handle, wherein the proximal end of the shaft is positioned in the handle, the shaft including a lumen extending from the distal end to the proximal end; and a seal assembly positioned in the handle and coupled to the proximal end of the shaft, the seal assembly including a seal housing, a seal disposed within the seal housing, and a seal compressor member coupled to the seal housing and configured to compress the seal within the seal housing in an axial direction.

In another representative example, a delivery apparatus comprises a handle having at least one window; a delivery shaft extending distally from the handle, the delivery shaft having a distal end and a proximal end, wherein the proximal end of the delivery shaft is positioned within the handle; a spine assembly positioned at least partially around the delivery shaft, the spine assembly coupled to the handle; an adjustment mechanism configured to adjust a curvature of the distal end of the delivery shaft; and an indicator configured to indicate an amount of adjustment by the adjustment mechanism, the indicator positioned within the handle and viewable through the at least one window.

In another representative example, an assembly comprises a delivery apparatus including the handle of any example herein; and a prosthetic implant releasably coupled to the delivery apparatus.

In another representative example, an assembly comprises the delivery apparatus of any example herein; and a prosthetic implant releasably coupled to the delivery apparatus.

In another representative example, a method for implanting a prosthetic implant comprises inserting a delivery apparatus into vasculature of a patient, wherein the delivery apparatus comprises the delivery apparatus of any example herein; and causing a first slider to slide relative to a first background member within a handle of the delivery apparatus, wherein the slider indicates an amount of a first type of adjustment of a shaft of the delivery apparatus.

In another representative example, a method for implanting a prosthetic implant comprises inserting a shaft of a delivery apparatus into vasculature of a patient, wherein the delivery apparatus includes a handle external to the patient, wherein the handle comprises the handle of any example herein; and rotating the delivery apparatus relative to the vasculature of the patient, wherein the handle comprises a support member configured to transmit torque from the handle to a distal end of the shaft.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side view of a delivery apparatus configured to deliver a prosthetic medical device to a target implantation site of a patient, according to one example.

FIG. 6 is a cross-sectional side view of the delivery apparatus of FIG. 5.

FIG. 7 is a perspective view of the delivery apparatus of FIG. 5 with a distal portion of housing removed to illustrate internal components of the delivery apparatus.

FIG. 8 is a perspective view of a spine extension of the delivery apparatus of FIG. 5, according to one example.

FIG. 9 is a perspective view of the distal portion of housing of the delivery apparatus of FIG. 5, according to one example.

FIG. 10 is a proximal end view of the spine extension of FIG. 8 positioned within the distal portion of housing of FIG. 9.

FIG. 11 is a perspective view of the delivery apparatus of FIG. 5 with the distal portion of housing and the spine extension removed to illustrate a shaft of the delivery apparatus.

FIG. 12 is another perspective view of the delivery apparatus of FIG. 5 with the distal portion of housing removed.

FIG. 13 is a perspective view of a spine assembly of the delivery apparatus of FIG. 5 positioned around a shaft of the delivery apparatus of FIG. 5, the spine assembly including the spine extension of FIG. 9.

FIG. 14 is a perspective view of a distal spine of the spine assembly of FIG. 13.

FIG. 15 is a perspective view of a proximal spine of the spine assembly of FIG. 13.

FIG. 16 is a perspective view of components of an adjustment mechanism positioned on the spine assembly of FIG. 13.

FIG. 17 is a perspective view of additional components of the adjustment mechanism positioned on the spine assembly of FIG. 13.

FIG. 18 is a perspective view of an upper segment of an intermediate portion of housing of the delivery apparatus of FIG. 5.

FIG. 19 is a perspective view of a lower segment of the intermediate portion of housing of the delivery apparatus of FIG. 5.

FIG. 20 is a top view of an intermediate section of the delivery apparatus of FIG. 5 with the upper segment of the intermediate portion of housing of FIG. 18 removed.

FIG. 21 is a perspective view of the intermediate section of the delivery apparatus of FIG. 5 with the upper segment of the intermediate portion of housing of FIG. 18 removed to illustrate components of an indicator assembly.

FIG. 22 is a perspective view of the intermediate section of the delivery apparatus of FIG. 5 with the upper segment of the intermediate portion of housing of FIG. 18 and windows of the indicator assembly removed.

FIG. 23 is another perspective view of the delivery apparatus of FIG. 5.

FIG. 24 is a perspective view of the delivery apparatus of FIG. 5 with a proximal portion of housing removed to illustrate internal components of the delivery apparatus.

FIG. 25 is a cross-sectional side view of a proximal section of the delivery apparatus of FIG. 5.

FIG. 26 is a perspective view of a seal housing of the delivery apparatus of FIG. 5, according to one example.

FIG. 27 is a perspective view of a seal compressor member of the delivery apparatus of FIG. 5, according to one example.

FIG. 28 is a perspective view of a seal assembly of the delivery apparatus of FIG. 5 including the seal housing of FIG. 26 and the seal compressor member of FIG. 27, according to one example.

FIG. 29 is a side view of another delivery apparatus configured to deliver a prosthetic medical device to a target implantation site of a patient, according to one example.

FIG. 30 is a side view of an example delivery system including the delivery apparatus of FIG. 29.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises. ” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined. As used herein, other directional terms (e.g., “vertical,” “horizontal,” etc.) refer to a direction relative to the reference frame of the figures and is not meant to be limiting with respect to potential alternative orientations.

### Introduction to the Disclosed Technology

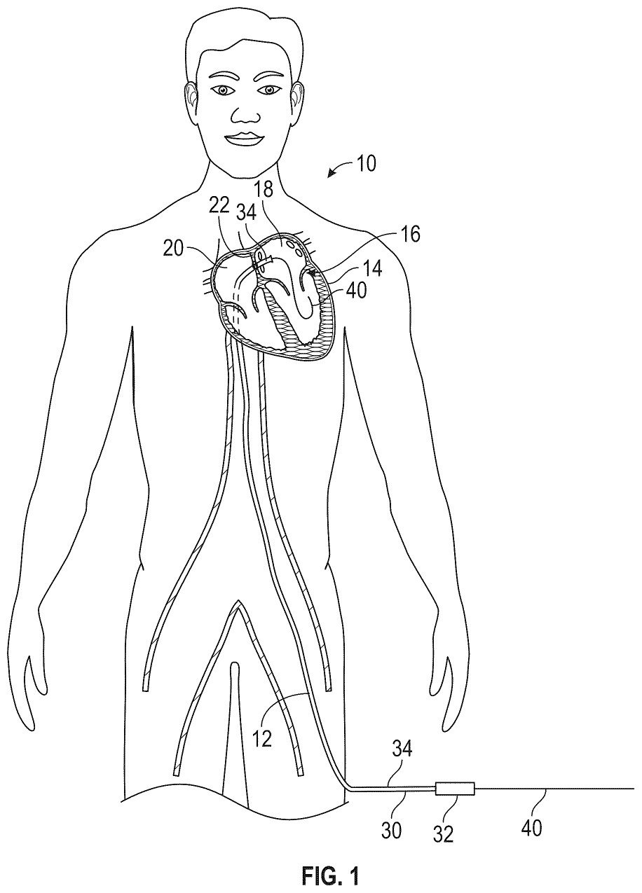

Described herein are various systems, apparatuses, methods, or the like, that, in some examples, can be used in or with delivery apparatuses for prosthetic medical devices (such as prosthetic heart valves or docking devices). In some examples, the delivery apparatuses disclosed herein can be used to deliver a docking device for a transcatheter prosthetic heart valve into the vasculature of a patient at a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus and then the prosthetic heart valve delivery apparatus is used to deliver a transcatheter prosthetic heart valve inside the docking device.

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve.

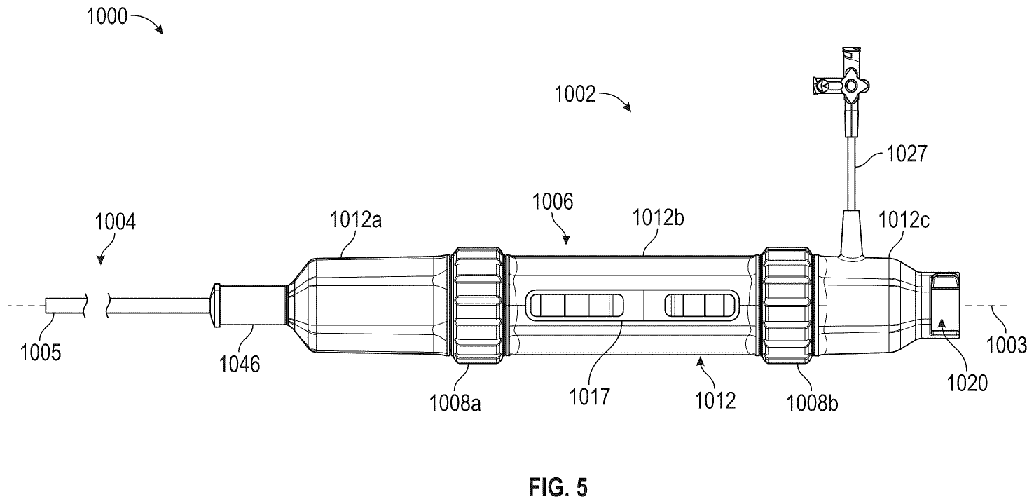

Exemplary delivery apparatuses for delivering a docking device at a native heart valve are shown in more detail in FIGS. 5-30. In some examples, as shown in FIGS. 5-22, a delivery apparatus can include a spine assembly within a handle of the delivery apparatus that is configured to transmit torque exerted on the handle to a shaft of the delivery apparatus.

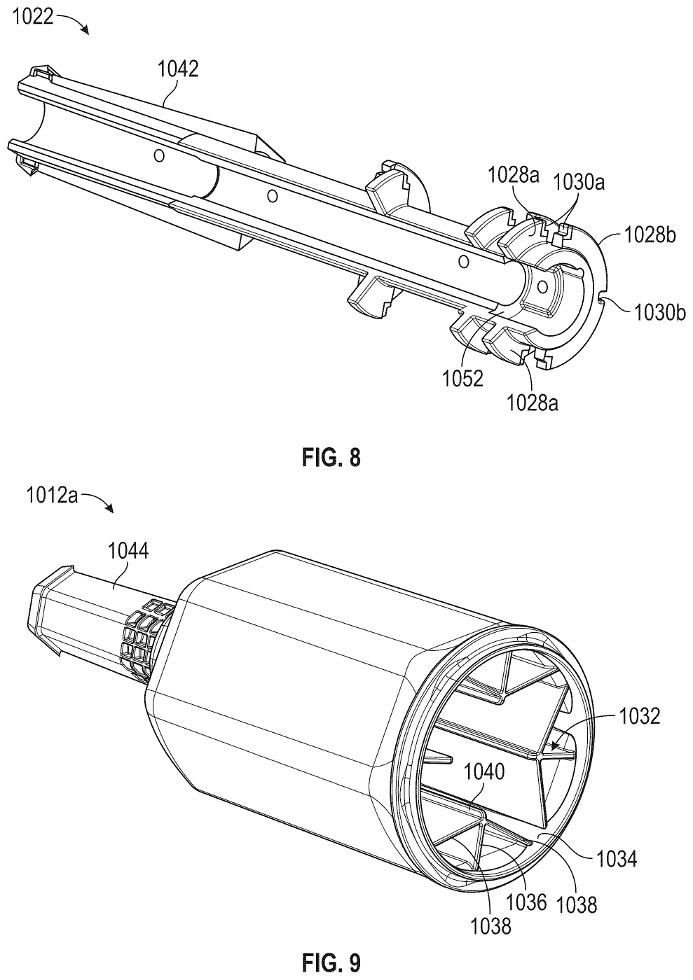

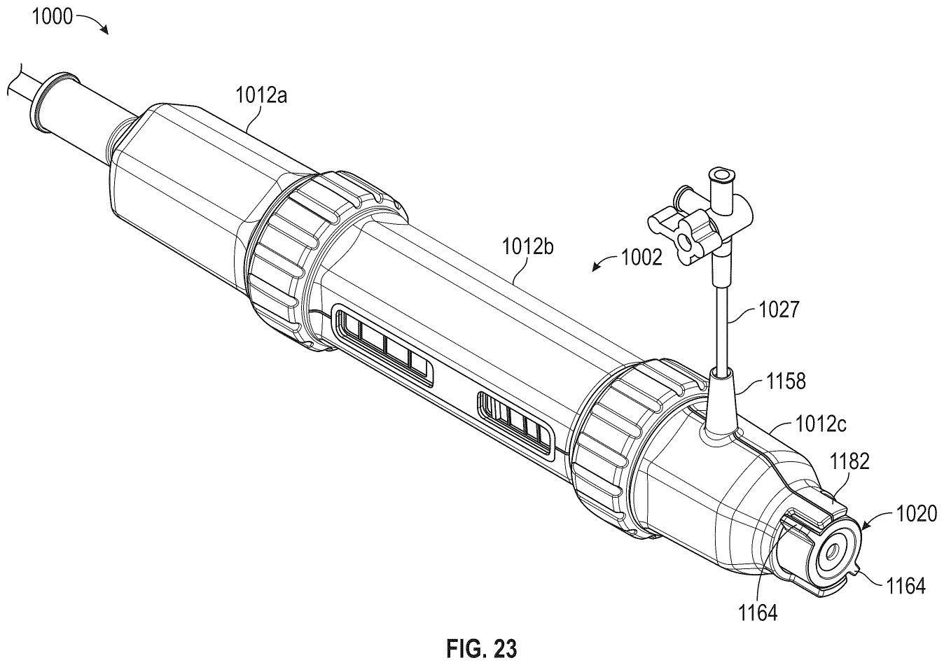

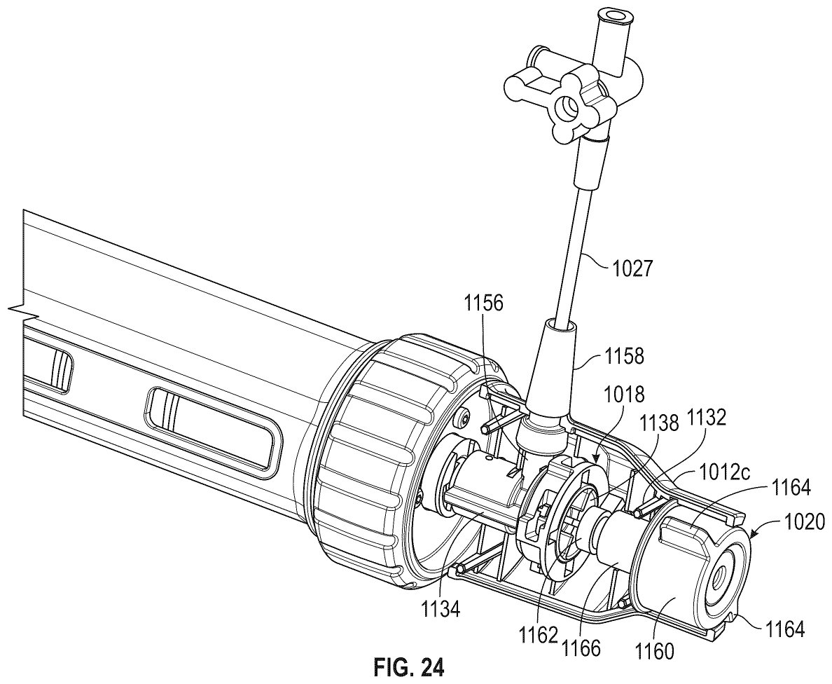

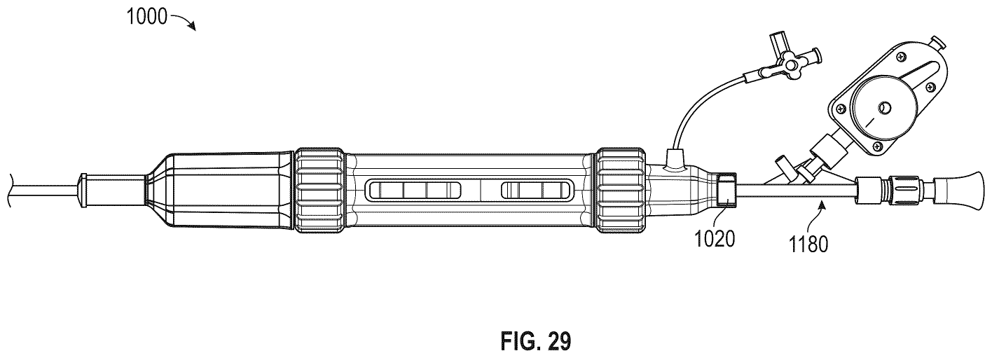

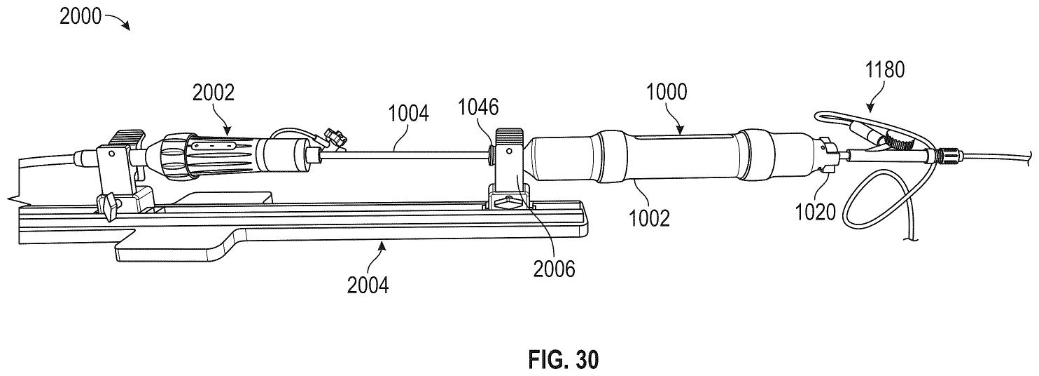

Additional details of an exemplary spine assembly are shown in FIGS. 8 and 13-15. The delivery apparatus can also include an adjustment mechanism to manipulate (e.g., control, steer, flex, etc.) the distal end of the shaft. Additional details of an exemplary spine assembly are shown in FIGS. 11-12 and 16-17. In some examples, as shown in FIGS. 5 and 20-22, the handle of the delivery apparatus can include an indicator to visually indicate manipulation of the distal end of the shaft. The delivery apparatus can also include a seal assembly including one or more seals that can be uniformly compressed in an axial direction (e.g., without the use of fasteners such as screws or bolts, etc.), as shown in FIGS. 24-28. Additionally, the delivery apparatus can include a locking mechanism configured to lock a device inserted through the delivery apparatus (FIGS. 29-30), such that the device is selectively prevented from moving relative to the delivery apparatus. Additional details of an exemplary locking mechanism are shown in FIGS. 23-25.

### Examples of the Disclosed Technology

FIGS. 1-4 depict an exemplary transcatheter heart valve replacement procedure (e.g., a mitral valve replacement procedure) which utilizes prosthetic implants including a docking device 52 and a prosthetic heart valve 62, according to one example. During the procedure, a user first creates a pathway to a patient's native heart valve using a guide catheter 30 (FIG. 1).

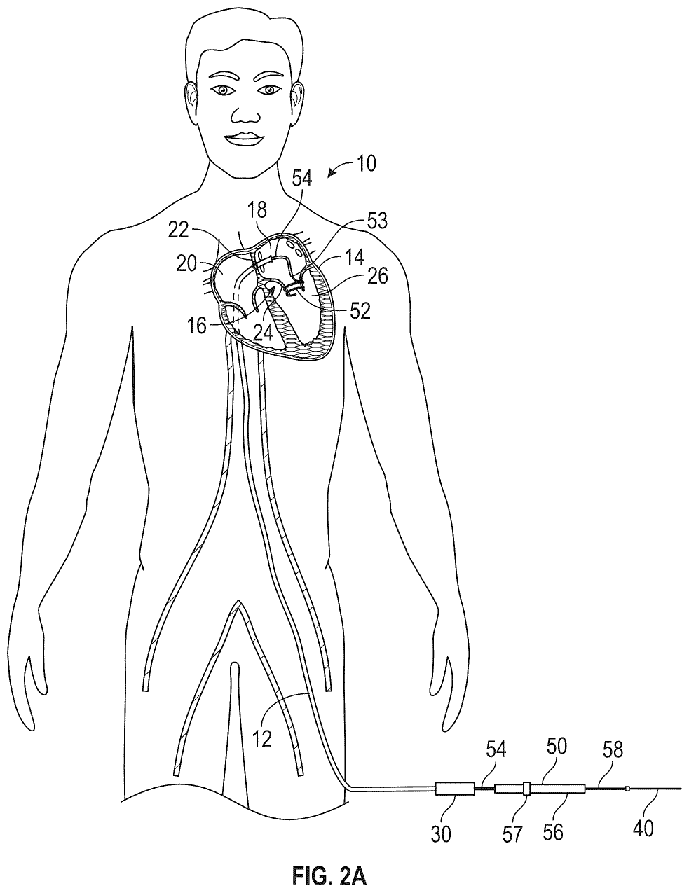

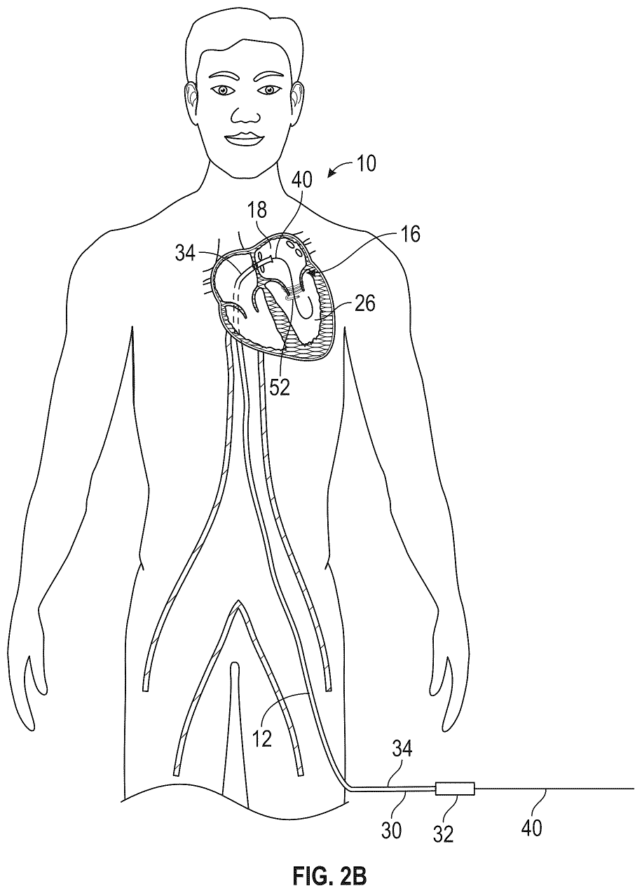

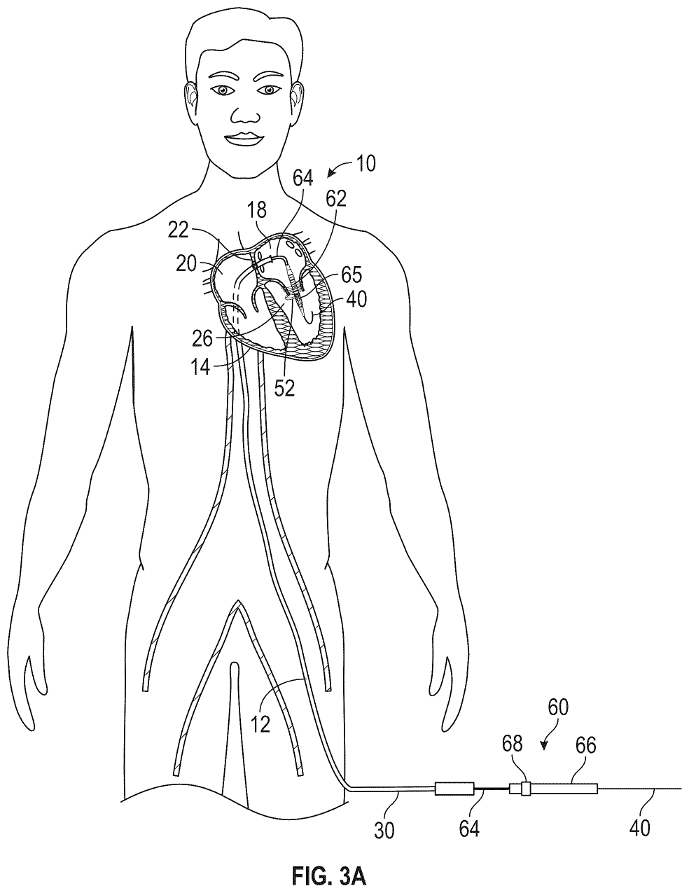

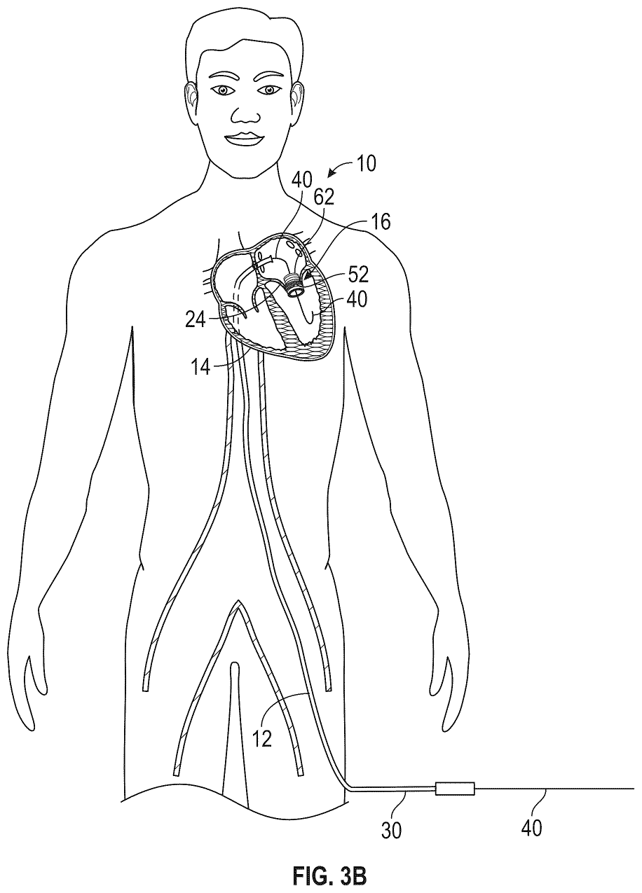

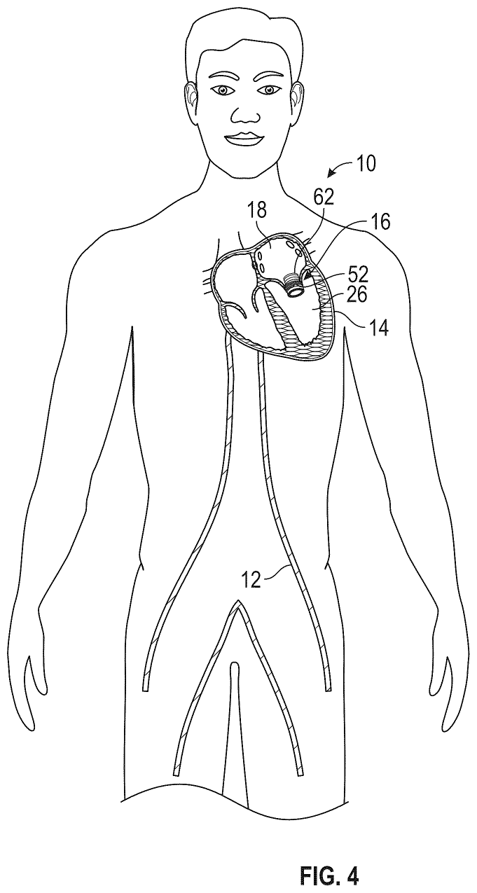

The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter”and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in positioning the delivery shaft 54 within the heart 14. For example, the one or more articulation members 57 can comprise one or more of knobs, buttons, wheels, and/or other types of physically adjustable control members that are configured to be adjusted by the user to flex, bend, twist, turn, and/or otherwise articulate the distal end portion 53 of the delivery shaft 54 to aid in positioning the delivery shaft 54 within the heart 14 for deployment of the docking device 52 at the implantation site (e.g., the native mitral valve 16).

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. In other examples, the guidewire 40 can be fully removed from the guide catheter 30 prior to insertion of the docking device delivery apparatus 50. The user may then continue to advance the delivery shaft 54 of the docking device delivery apparatus 50 through the blood vessel 12 within the guide catheter 30 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10, such that only the guide catheter 30 remains inside the patient 10. In some examples, both the guide catheter 30 and the guidewire 40 remain inside the patient 10. After removing the docking device delivery apparatus 50, the guidewire 40 can be advanced through and/or out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66.

The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Certain examples are directed to delivery systems and/or apparatuses to deliver prosthetic medical devices (such as the docking device 52 and/or the prosthetic heart valve 62 described above with reference to FIGS. 1-4) to a heart and/or native valve of an animal, human, cadaver, cadaver heart, anthropomorphic ghost, and/or simulation/simulator. Such devices include transcatheter devices that can be used to guide the delivery of a docking device through vasculature.

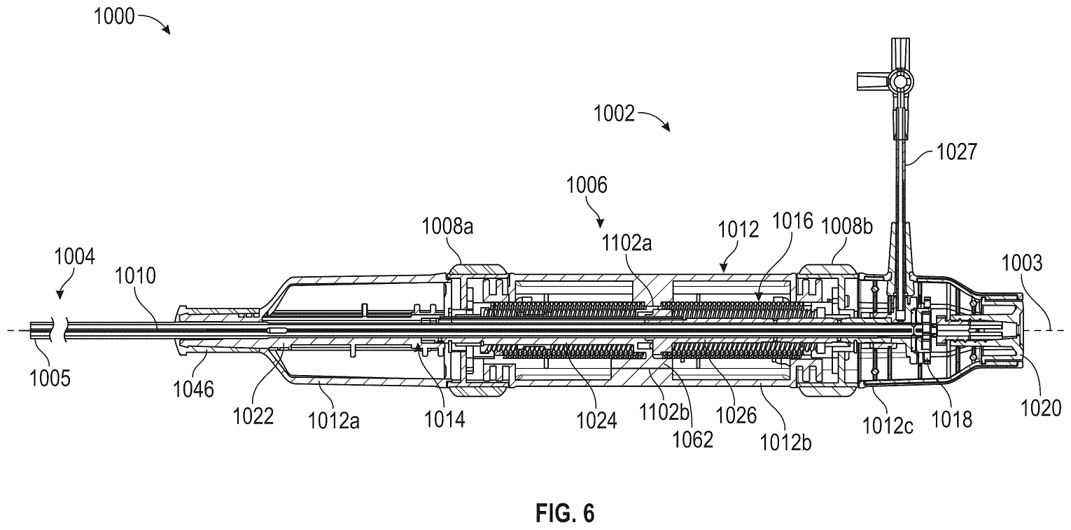

An example delivery apparatus 1000 configured to deliver a docking device to a target implantation site is shown in FIGS. 5-6. In some examples, the delivery apparatus 1000 can be used as the docking device delivery apparatus 50 in a prosthetic valve implantation procedure, as described above with reference to FIGS. 1-4. The delivery apparatus 1000 can also be referred to as a “docking device delivery apparatus,” “dock delivery apparatus,” “dock delivery catheter,” or “dock delivery system.”The delivery apparatus 1000 can include a handle assembly 1002 and a delivery shaft 1004 (also referred to as the “delivery catheter,” “outer shaft,” “delivery sheath,” or “outer sheath”) extending distally from the handle assembly 1002. The delivery shaft 1004 can be coaxial with a central longitudinal axis 1003 of the delivery apparatus 1000. The handle assembly 1002 can include a handle 1006 including one or more knobs, buttons, wheels, and/or other means for controlling and/or actuating one or more components of the delivery apparatus 1000. For example, in some examples, as shown in FIG. 5, the handle 1006 can include knobs 1008a and 1008b which can be configured to steer or control flexing of the delivery apparatus 1000 (e.g., the delivery shaft 1004, etc.).

The delivery shaft 1004 has a main (or primary) lumen 1010 that is defined by an inner surface of a wall of the delivery shaft 1004 (FIG. 6). The main lumen 1010 is configured to receive one or more devices therein (such as any of the docking devices and the pusher assemblies described herein). In some examples, as shown in FIG. 6, the delivery shaft 1004 can extend into the handle 1006. Further, in some instances, the main lumen 1010 can extend through the handle 1006 to a locking mechanism 1020 disposed at a proximal end of the handle 1006. For example, in these instances, an inner surface of the locking mechanism 1020 can comprise a lumen coaxial with the main lumen 1010 of the delivery shaft 1004. Thus, the delivery apparatus 1000 comprises a lumen extending from the locking mechanism 1020 to a distal end portion 1005 of the delivery shaft 1004.

In certain examples, the handle 1006 can also include an outer housing 1012. Within the housing 1012, the handle 1006 can include a spine assembly 1014, an adjustment mechanism 1016 including the knobs 1008a, 1008b, an indicator assembly 1017, a seal assembly 1018, and a locking mechanism 1020. In some instances, the housing 1012 can be integrally formed as a single, unitary component. In other instances, as depicted, the housing 1012 can comprise one or more segments that are formed as separate components that are coupled together (e.g., via fasteners, adhesive, mating features, and/or other means for coupling). For example, the housing 1012 can comprise a distal segment 1012a, an intermediate segment 1012b that is proximal to the distal segment 1012a, and a proximal segment 1012c that is proximal to the intermediate segment 1012b. In some examples, the housing 1012 can be manufactured using one or more molding processes (e.g., injection molding, etc.).

In the depicted example, the distal segment 1012a of the housing 1012 (which can also be referred to as the “nose cone”) is distal to the knob 1008a and can include a portion of the spine assembly 1014, as described in more detail below. The intermediate segment 1012b of the housing 1012 (which can also be referred to as the “main housing”) is positioned between the knobs 1008a, 1008b. The main housing 1012b can also include a portion of the spine assembly 1014 as well as the adjustment mechanism 1016 and the indicator assembly 1017, as described in more detail below. The proximal segment 1012c of the housing 1012 (which can also be referred to as the “proximal housing”) is proximal to the knob 1008b and can include the seal assembly 1018 and the locking mechanism 1020, as described in more detail below. In some instances, the portion of the handle 1006 that is proximal to the nose cone 1012a (e.g., the main housing 1012b, the proximal housing 1012c, and/or the components included therein, etc.) can also be referred to herein as the “body”of the handle 1006.

The spine assembly 1014 can be configured to partially cover (e.g., surround) the delivery shaft 1004. The spine assembly 1014 can support the delivery shaft 1004 as torque is applied to the handle 1006. Specifically, the spine assembly 1014 can be connected to the housing 1012 and can transfer torque exerted on the handle 1006 from the housing 1012 of the handle 1006 to the delivery shaft 1004, including to a distal end portion 1005 of the delivery shaft 1004. Accordingly, in some examples, the spine assembly 1014 can also be referred to as a support member.

In some instances, the spine assembly 1014 can be integrally formed as a single, unitary component. In other instances, as depicted, the spine assembly 1014 can comprise one or more segments that are formed as separate components that are coupled together (e.g., via fasteners, adhesive, mating features, and/or other means for coupling). For example, the spine assembly 1014 can include a spine extension 1022, a distal spine 1024 and a proximal spine 1026. The spine extension 1022 (also referred to herein as a “nose bridge”) is connected to the distal spine 1024 and extends distally therefrom. The proximal spine 1026 is connected to the distal spine 1024 and extends proximally therefrom. As shown in FIG. 6, the spine extension 1022 is positioned within the nose cone 1012a and the distal spine 1024 and the proximal spine 1026 are positioned within the main housing 1012b. It should be appreciated that in some examples, the distal spine 1024 and the proximal spine 1026 can be integrally formed as a single, unitary component. In some instances, the distal spine 1024 and the proximal spine 1026 can be collectively referred to as a “spine” or “main spine.”The handle assembly 1002 can also include a flush tube 1027 connected to the housing 1012, distal to a seal of the seal assembly 1018. In some examples, as depicted, the flush tube 1027 is connected to the proximal housing 1012c.

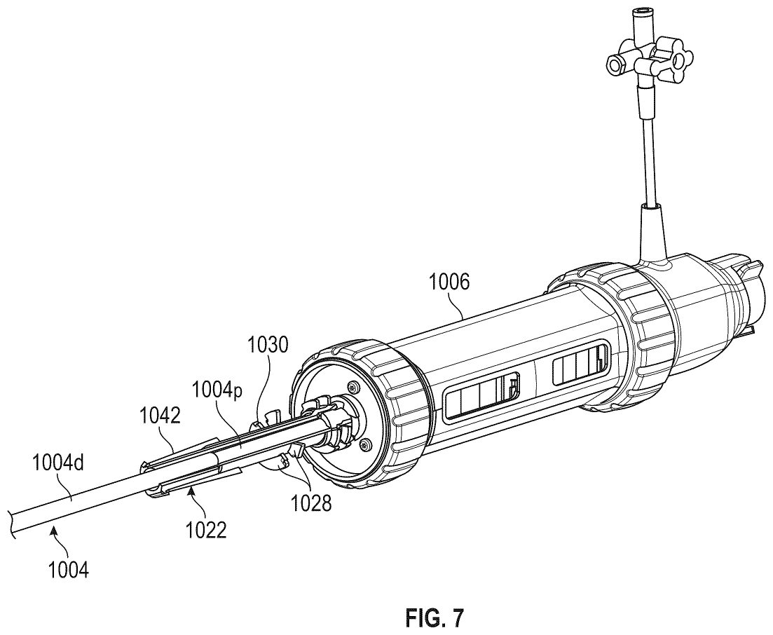

FIG. 7 depicts the delivery apparatus 1000 with the nose cone 1012a removed. As shown, the spine extension 1022 can extend distally from the handle 1006 along a length of the delivery shaft 1004. The spine extension 1022 can partially surround (e.g., partially cover) the delivery shaft 1004 in a circumferential direction. As described in more detail below, the delivery shaft 1004 can include a distal section 1004d and a proximal section 1004p. In some examples, a strengthening braid can be included in the distal section 1004d of the delivery shaft 1004 to help strengthen the delivery shaft 1004 and transfer torque to the distal end portion 1005 of the delivery shaft 1004. As shown, the distal section 1004d can have a relatively larger outer diameter (e.g., due to the presence of the strengthening braid, etc.) and the proximal section 1004p can have a relatively smaller outer diameter. The spine extension 1022 can partially surround the delivery shaft 1004 at the transition from the distal section 1004d to the proximal section 1004p to provide added support and strength for the delivery shaft 1004, particularly the proximal section 1004p of the delivery shaft 1004.

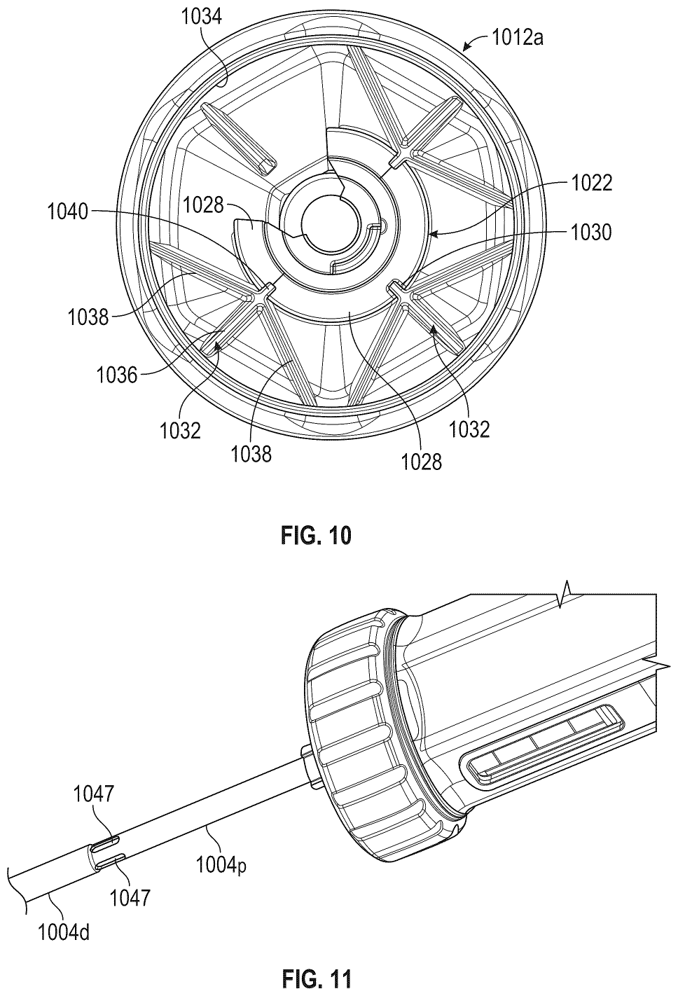

The spine extension 1022 can include radial projections 1028 that are spaced apart from each other in an axial direction along a length of the spine extension 1022. The radial projections 1028 of the spine extension 1022 can be configured to mate with the nose cone 1012a (FIG. 10). In some examples, as depicted, the radial projections 1028 can include mating features or elements 1030, such as slots, notches, or the like. The mating elements 1030 can be configured to engage with internal mating elements 1032 of the nose cone 1012a (FIG. 9).

It should be appreciated that each radial projection 1028 can include one or more mating elements 1030 of the same or different configurations. For example, as best shown in FIG. 8, a first radial projection 1028a can include one notch 1030a and a second radial projection 1028b can include two notches 1030a and one slot 1030b. In some instances, as depicted, notches 1030a on axially-adjacent radial projections 1028a and 1028b can form corresponding halves of a slot. As shown, the radial projection 1028b can be spaced apart from the radial projection 1028a in a circumferential direction and in an axial direction. In some examples, more than one radial projection 1028a can be located at the same axial position. As a result, the radial projections 1028a are only spaced apart in the circumferential direction. It should be appreciated that radial projections 1028 can be configured with other configurations of mating elements 1030 and/or other spacing (e.g., in only the axial direction, etc.).

As shown in FIG. 9, the nose cone 1012a can include mating elements 1032 which project from an inner surface 1034 of the nose cone 1012a. The mating elements 1032 can extend along a length of the inner surface 1034 of the nose cone 1012a in an axial direction. In this manner, the mating elements 1032 can also be referred to as “axial runners.” In the illustrated example, the nose cone 1012a includes three axial runners 1032. It should be appreciated that the nose cone 1012a can include a different number of axial runners 1032 (e.g., fewer than three axial runners 1032, more than three axial runners 1032, etc.). As shown, each axial runner 1032 can include a primary segment or panel 1036 which projects in a radial direction from the inner surface 1034. Each axial runner 1032 can also include two secondary segments or panels 1038 which can project from the inner surface 1034 at an angle and can intersect with or contact the primary segment 1036. Each tip 1040 of the primary segment 1036 can extend beyond the secondary segments 1038 in the radial direction (e.g., towards the center of the nose cone 1012a, etc.). Each tip 1040 can be configured to engage with an axial slot or groove defined by the mating elements 1030 of the spine extension 1022.

The axial runners 1032 can be configured to mate with the mating elements 1030 of the spine extension 1022. As shown in FIG. 10, the mating elements 1030 of each radial projection 1028 can be axially aligned and can correspond with the axial runners 1032 of the nose cone 1012a. This alignment allows the spine extension 1022 to be inserted into the nose cone 1012a and to be moved axially with respect to the nose cone 1012a (e.g., during assembly of the delivery apparatus 1000, etc.). Additionally, the axial runners 1032 can be configured to prevent rotational movement of the nose cone 1012a relative to the spine extension 1022. In this way, torque exerted on the nose cone 1012a can be transferred from the nose cone 1012a to the spine extension 1022 via the radial projections 1028 and the axial runners 1032. Further, the spine extension 1022 can be configured to transmit the torque to the delivery shaft 1004.

Referring again to FIGS. 8-9, the spine extension 1022 can include a distal end portion 1042 which partially surrounds the delivery shaft 1004 in a circumferential direction (e.g., surrounds half of the circumference of the delivery shaft 1004, etc.). The nose cone 1012a can also include a distal end portion 1044 which partially surrounds the delivery shaft 1004 in a circumferential direction (e.g., surrounds the other half of the circumference of the delivery shaft 1004, etc.). Thus, when the nose cone 1012a is mated with the spine extension 1022, the distal end portion 1042 of the nose cone 1012a and the distal end portion 1044 of the spine extension 1022 can be aligned in an axial direction and can fully surround the delivery shaft 1004 in a circumferential direction. In some examples, a cylindrical cap 1046 can be positioned over the distal end portions 1042, 1044 of the spine extension 1022 and the nose cone 1012a (FIGS. 5-6). The cylindrical cap 1046 can be configured to prevent movement (e.g., axial movement, etc.) of the spine extension 1022 relative to the nose cone 1012a. In some instances, the cap 1046 can comprise an elastomeric material.

In some instances, as depicted, an outer surface of the nose cone 1012a can include six faces to define a hexagonal cross-section. The distal end portion 1042 of the nose cone 1012a can include three faces and the distal end portion 1044 of the spine extension 1022 can include three faces, such that the distal end portions 1042, 1044 can also define a hexagonal cross-section. The hexagonal cross-sections can help facilitate grip of the handle 1006. In other instances, the outer surface of the nose cone 1012a and/or the distal end portions 1042, 1044 can include a different number of faces, such that a different cross-sectional shape is defined (e.g., circular, square, octagonal, etc.). The other segments of the housing 1012 (e.g., main housing 1012b and proximal housing 1012c) can also the same cross-sectional shape (e.g., hexagonal, etc.) as the nose cone 1012a and/or a different cross-sectional shape to help facilitate grip of the handle 1006. In some instances, the cylindrical cap 1046 can include an inner shape (e.g., hexagon, etc.) that is complementary to the shape (e.g., hexagon, etc.) of the outer surfaces of the distal end portions 1042, 1044 to limit or prevent rotation of the cap 1046 rotate relative to the spine extension 1022 and the nose cone 1012a.

While the cap 1046 is shown as cylindrical in the illustrated examples, in other examples, the cap 1046 can comprise non-cylindrical shapes including, for example, hexagonal, square, octagonal, etc.

In lieu of or in addition to the cylindrical cap 1046, the delivery apparatus 1000 can comprise one or more other components configured to couple the spine extension 1022 to the nose cone 1012a in some examples. For example, the spine extension 1022 and/or the nose cone 1012a can include mating features configured to mate the spine extension 1022 with the nose cone 1012a to prevent relative movement therebetween.

As introduced above, the distal end portion 1005 of the delivery shaft 1004 can be configured to be steerable via the knobs 1008a, 1008b and the adjustment mechanism 1016. In one example, by rotating a knob (e.g., 1008a or 1008b) on the handle 1006, a curvature of the distal end portion 1005 can be adjusted so that the distal end portion 1005 of the delivery shaft 1004 can be oriented in a desired angle. For example, to implant a docking device (e.g., docking device 52) at the native mitral valve location, the distal end portion 1005 of the delivery shaft 1004 can be steered so that the docking device can be positioned at a target implantation location.

In some examples, the knob 1008a can be coupled to a first pull wire (not shown) of the adjustment mechanism which can control the front flex of the delivery shaft 1004 (e.g., based on the tension of the first pull wire, etc.). The knob 1008b can be coupled via the adjustment mechanism 1016 to a second pull wire (not shown) which can control the back flex of the delivery shaft 1004 (e.g., based on the tension of the second pull wire, etc.). The first and second pull wires can be connected to the distal end portion 1005 of the delivery shaft 1004 and extend proximally into the handle 1006 (e.g., into the main housing 1012b of the handle 1006, etc.).

Referring now to FIG. 11, in some examples, it is necessary for the first and second pull wires to pass from a location inside of or within the delivery shaft 1004 to a location outside of the delivery shaft 1004. As such, the proximal section 1004p of the delivery shaft 1004 can include two axially extending slots 1047 (also referred to as “openings”) which are configured to allow the first and second pull wires to exit the location inside of or within the distal section 1004d of the delivery shaft 1004 and pass along the outside of the proximal section 1004p of the delivery shaft 1004. Because the pull wires must pass from a location inside of or within the delivery shaft 1004 to a location outside of the delivery shaft 1004, the strengthening braid included in the distal section 1004d of the delivery shaft 1004 cannot extend along the entire length of the delivery shaft 1004. As such, the spine extension 1022 can provide additional strength to the delivery shaft 1004, while allowing the pull wires to pass through the slots 1047 of the delivery shaft 1004.

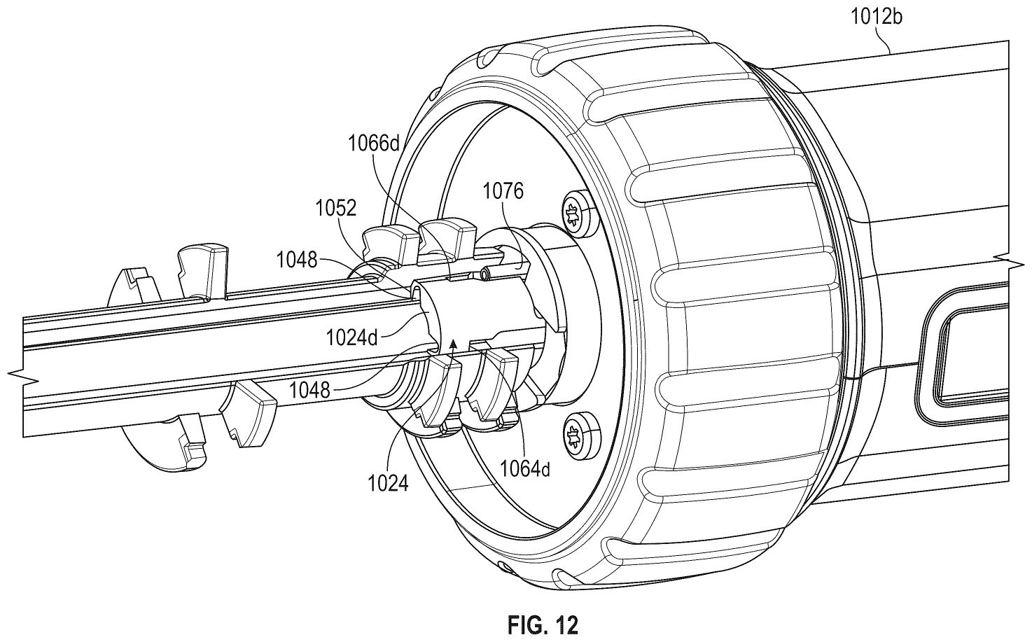

Following the pull wires in a proximal direction, after the pull wires pass through the slots 1047, the first and second pull wires can pass through openings 1048 in a distal end 1024d of the distal spine 1024, as shown in FIG. 12. After passing through the openings 1048, the pull wires can connect to other components of the adjustment mechanism 1016 which can be located within the main housing 1012b of the handle 1006, as described in more detail below.

The distal end 1024d of the distal spine 1024 can be coupled to the spine extension 1022. Specifically, the inner surface of the spine extension 1022 can include a step 1052 defining a seat for the distal end 1024d of the distal spine 1024 (see also FIG. 8). For example, during assembly of the spine assembly 1014, the distal spine 1024 may be inserted into the spine extension 1024 and moved axially relative to the spine extension 1022 until the distal end 1024d contacts the step 1052 of the spine extension 1022. In this way, the spine extension 1022 can partially surround the distal end 1024d of the distal spine 1024, such that the distal end 1024d of the distal spine 1024 is positioned within the spine extension 1022.

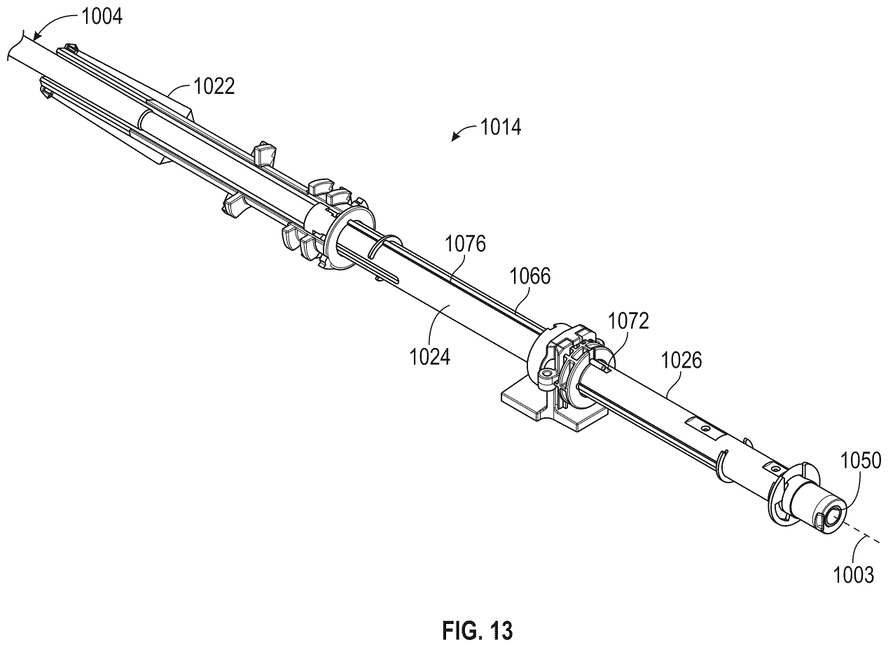

FIG. 13 illustrates the delivery shaft 1004 positioned within a central lumen 1050 of the spine assembly 1014. The central lumen 1050 can be coaxial with the central longitudinal axis 1003 and can be defined by inner surfaces of the spine extension 1022, the distal spine 1024, and the proximal spine 1026.

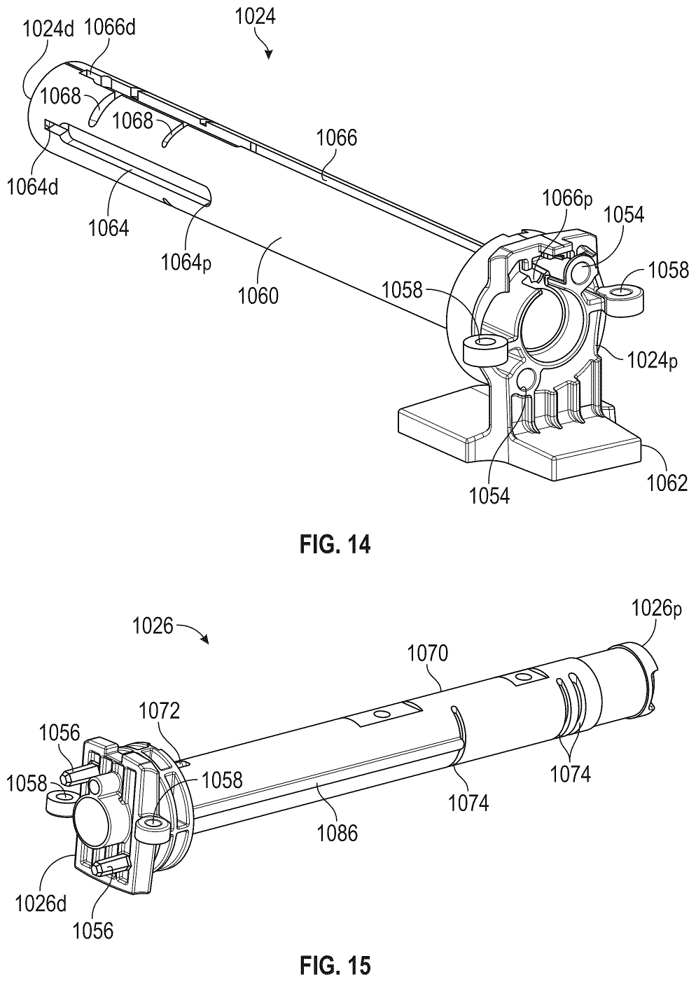

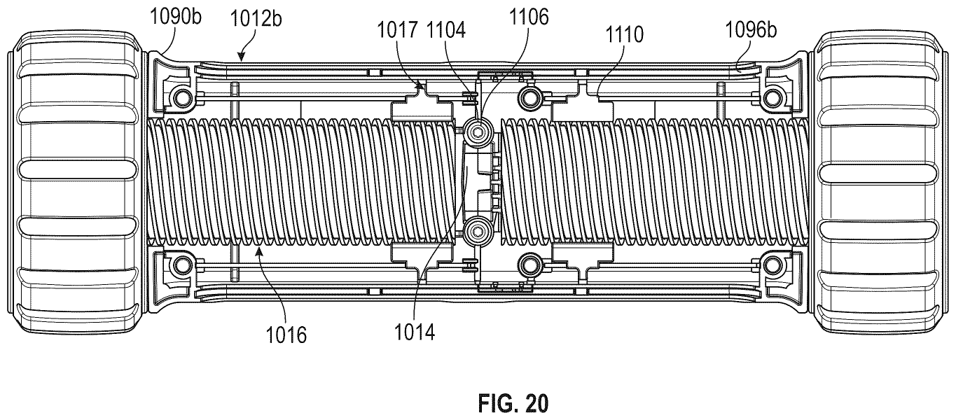

The distal spine 1024 can be coupled to the proximal spine 1026. As shown in FIGS. 14-15, the distal spine 1024 can include mating features 1054 (e.g., sockets, etc.) which correspond to mating features 1056 (e.g., pins, etc.) of the proximal spine 1026. The distal spine 1024 and the proximal spine 1026 can also include holes 1058 which can be configured to couple the spine assembly 1014 to the housing 1012. Specifically, when the distal spine 1024 is coupled to (e.g., mated with, etc.) the proximal spine 1026, the holes 1058 of the distal spine 1024 and the proximal spine 1026 can align such that fasteners (e.g., fasteners 1106 shown in FIG. 20, screws, bolts, etc.) can be inserted through the holes 1058 to attach the spine assembly 1014 to the main housing 1012b.

The distal spine 1024 can include a spine shaft 1060 and a base 1062. The spine shaft 1060 can be generally cylindrical and can be parallel to the central longitudinal axis 1003 of the delivery apparatus 1000. The base 1062 can be connected to the spine shaft 1060 and can be radially spaced or offset from the spine shaft 1060. In the illustrated example, the base 1062 is located at a proximal end 1024p of the distal spine 1024. The base 1062 can be positioned within the main housing 1012b and contact an inner surface of the main housing 1012b, as described in more detail below. In some instances, the base 1062 can be the only component of the spine assembly 1014 that contacts the housing 1012 (e.g., apart from fasteners 1106 inserted through holes 1058, etc.). As such, the base 1062 can be configured to align the spine assembly 1014 relative to the housing 1012, such that the central lumen 1050 of the spine assembly 1014 is coaxial with the central longitudinal axis 1003 of the delivery apparatus 1000. In this manner, the base 1062 can create an alignment datum (e.g., radial, vertical, axial, etc.) for the handle assembly 1002. In some instances, the holes 1058 of the distal spine 1024 and/or the proximal spine 1026 can also contact the housing 1012 (e.g., at bores 1104 shown in FIGS. 19-20, etc.).

The distal spine 1024 can include a first slot 1064 having a distal end 1064d and a proximal end 1064p. The distal spine 1024 can also include a second axially extending slot 1066 having a distal end 1066d and a proximal end 1066p. The first slot 1064 can be circumferentially spaced apart from the second slot 1066. In the illustrated example, the first slot 1064 and the second slot 1066 are circumferentially spaced apart by 90 degrees. As best shown in FIG. 12, the distal end 1064d of the first slot 1064 and the distal end 1066d of the second slot 1066 can be positioned distal to the main housing 1012b, such that the distal ends 1064d, 1066d are positioned within the nose cone 1012a.

Referring again to FIG. 14, the first slot 1064 can extend axially along a length of the spine shaft 1060 (e.g., less than the entire length of the spine shaft 1060, etc.). In particular, the proximal end 1064p of the first slot 1064 can be spaced apart in an axial direction from the proximal end 1024p of the spine 1024 (e.g., the proximal end 1064p of the first slot 1064 is distal to the proximal end 1024p of the spine 1024, etc.). The proximal end 1066p of the second slot 1066 can be positioned at or adjacent the proximal end 1024p of the spine 1024. In this way, the first slot 1064 can have a shorter axial length than the second slot 1066.

The spine shaft 1060 of the distal spine 1024 can also include radial grooves 1068 configured to receive clips, spacers, or the like. The radial grooves 1068 can be spaced apart along a length of the spine shaft 1060.

As shown in FIG. 15, the proximal spine 1026 can include a spine shaft 1070 that can be generally cylindrical and can be parallel to the central longitudinal axis 1003 of the delivery apparatus 1000. The spine shaft 1070 can include an axially extending slot 1072 positioned towards a distal end 1026d of the spine 1026. When the distal spine 1024 and the proximal spine 1026 are coupled (e.g., mated), the slot 1072 can be aligned with the second slot 1066 of the distal spine 1024. The spine shaft 1070 of the proximal spine 1026 can also include radial grooves 1074 configured to receive clips, spacers, or the like. The radial grooves 1074 can be spaced apart along a length of the spine shaft 1070 and can be positioned towards a proximal end 1026p of the spine 1026.

Although the distal spine 1024 is depicted as including the base 1062, it should be appreciated that in some examples, the proximal spine 1026 can include the base 1062 instead of the distal spine 1024. For example, the base 1062 can be connected to the spine shaft 1070 and can be radially spaced or offset from the spine shaft 1070. In these examples, the base 1062 can be located at the distal end 1026d of the proximal spine 1026.

The spine assembly 1014 can also include a wire shaft 1076, as shown in FIG. 13. In some instances, as depicted, the wire shaft 1076 can comprise a hollow cylindrical tube. The wire shaft 1076 can be positioned within the slot 1066 of the distal spine 1024 and the slot 1072 of the proximal spine 1026. One of the pull wires of the adjustment mechanism 1016 can be positioned within a lumen of the wire shaft 1076.

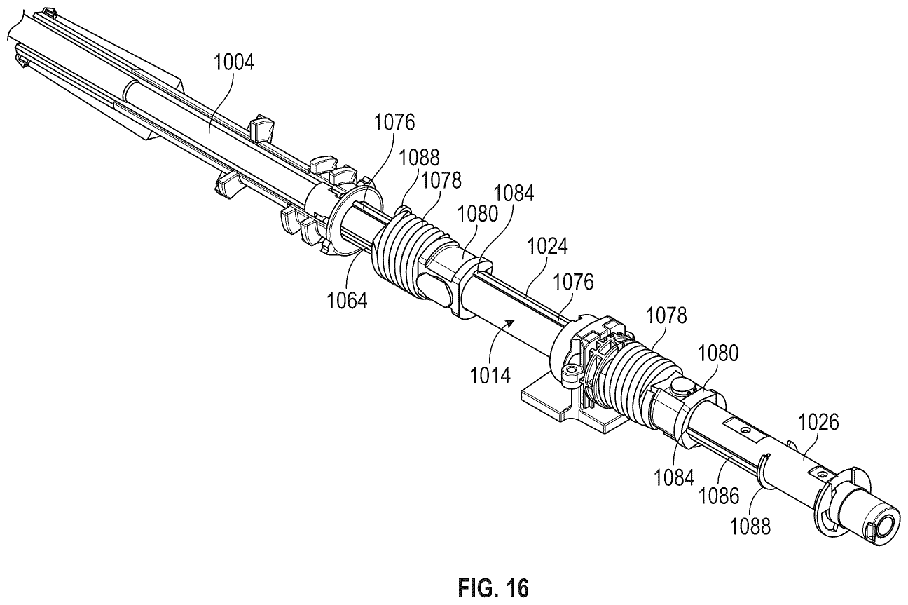

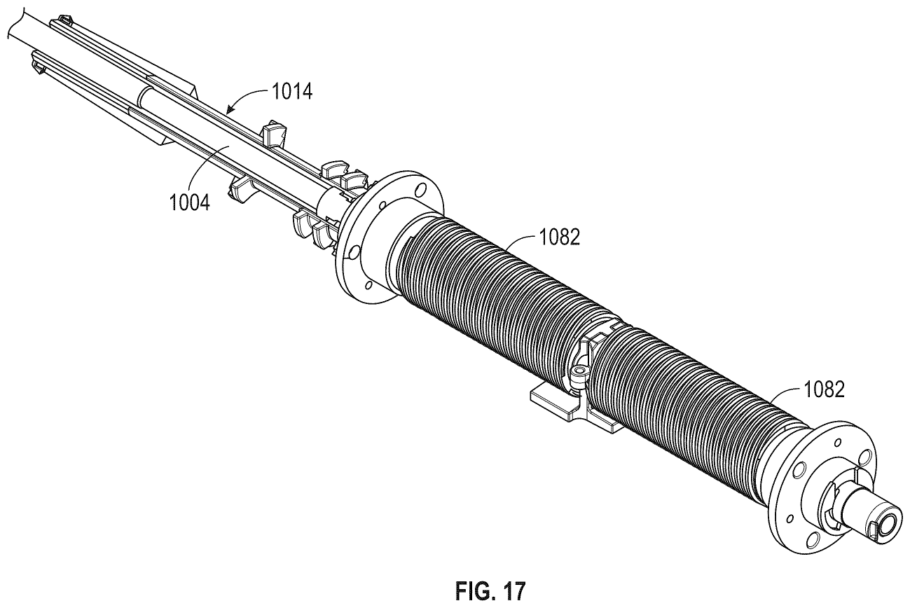

As introduced above, the adjustment mechanism 1016 (also referred to herein as a “flex assembly”) can be configured to steer the distal end portion 1005 of the delivery shaft 1004 via the knobs 1008a, 1008b and the pull wires (not shown) by increasing or decreasing the tension of the pull wires. In addition to the knobs 1008a, 1008b and the pull wires, the adjustment mechanism 1016 can also include slide nuts 1078, wire wraps 1080, and barrels 1082, as shown in FIGS. 16-17. The slide nuts 1078 can be positioned around the spine assembly 1014 and are configured to move axially relative to the spine assembly 1014. Specifically, a first slide nut 1078 can be positioned around the distal spine 1024 and a second slide nut 1078 can be positioned around the proximal spine 1026. The slide nuts 1078 can include external threads that engage with internal threads of the barrels 1082.

Each wire wrap 1080 can be positioned adjacent and proximal to one of the slide nuts 1078. The wire wraps 1080 can be configured to secure a distal end of one of the pull wires thereto (e.g., by wrapping an end of the pull wire around the wire wrap 1080, etc.). As discussed with reference to FIG. 12, the pull wires are fixed to the distal end portion 1005 of the delivery shaft 1004 and can pass through openings 1048 in the distal end 1024d of the distal spine 1024. After passing through the openings 1048, one of the pull wires can be positioned within the slot 1064 of the distal spine 1024 and connect to the wire wrap 1080 positioned around the distal spine 1024. The other pull wire, after passing through the opening 1048, can pass through the wire shaft 1076 and connect to the wire wrap 1080 positioned around the proximal spine 1026.

The barrels 1082 can be coupled to the knobs 1008a, 1008b such that rotation of one of the knobs 1008a, 1008b results in rotation of one of the barrels 1082. To adjust the distal end portion 1005 of the delivery shaft, one of the knobs (e.g., 1008a or 1008b) can be rotated which in turn rotates its corresponding barrel 1082. Rotation of the barrel 1082 can drive the slide nut 1078 in an axial direction via the threaded engagement between the barrel 1082 and the slide nut 1078. As the slide nut 1078 moves axially, the slide nut 1078 can push the wire wrap 1080 in an axial direction which can change the tension of the pull wire attached to the wire wrap 1080, thus adjusting the curvature or flex of the distal end portion 1005 of the delivery shaft 1004.

Each wire wrap 1080 can include a notch 1084 which can be configured to prevent rotational movement of the wire wrap 1080 as the slide nut 1078 pushes the wire wrap 1080. In particular, the notch 1084 of the wire wrap 1080 located on the distal spine 1024 can engage with the wire shaft 1076 as the wire wrap 1080 is axially moved by the slide nut 1078. In this manner, the wire shaft 1076 can be configured as a guide for the wire wrap 1080 that prevents rotational movement of the wire wrap 1080, while allowing the wire wrap 1080 to move in an axial direction relative to the distal spine 1024. As shown in FIG. 16, the proximal spine 1026 can include one or more guides 1086 that can be molded into the proximal spine 1026 and can be configured to guide axial movement of the corresponding wire wrap 1080 relative to the proximal spine 1026, while preventing rotational movement of the wire wrap 1080 relative to the proximal spine 1026 (e.g., similar to the wire shaft 1076). The guide 1086 can be a projection from the outer surface of the spine shaft 1070 that extends along a length of the proximal spine 1026. Although not shown, in some examples, the distal spine 1024 can also include one or more guides 1086 in addition to the wire shaft 1076. The wire shaft 1076 and the guides 1086 can also be configured to guide movement of the slide nuts 1078 (e.g., allow axial movement and prevent rotational movement, etc.).

In some instances, the spine assembly 1014 can include stops 1088 that extend radially from the distal spine 1024 and the proximal spine 1026 to limit the axial movement of the slide nuts 1078 and the wire wraps 1080 relative to the spine assembly 1014. In some instances, as depicted, the stops 1088 (e.g., clips, etc.) can be removably coupled to the spine assembly 1014 and can be positioned around the spine assembly 1014. In this manner, the stops 1088 can also limit the amount of curvature or flex of the distal end portion 1005 of the delivery shaft 1004 (e.g., by limiting the amount of tension that can be applied to the pull wires, etc.). Further, the amount of curvature or flex can be visually indicated by the indicator assembly 1017, as described in more detail below.

Further details of steerable catheters, the adjustment mechanism, and its variants are described in U.S. Pat. No. 10,076,638, which is incorporated by reference herein in its entirety.

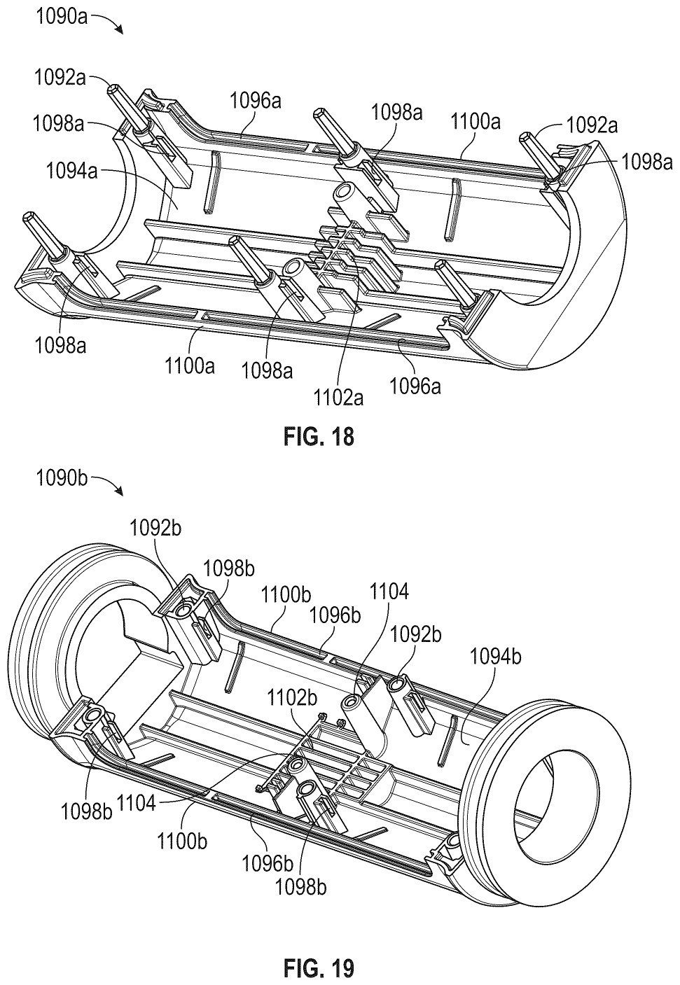

The slide nuts 1078, wire wraps 1080, and barrels 1082 of the adjustment mechanism 1016 can be disposed within the main housing 1012b. In some instances, the main housing 1012b can be integrally formed as a single, unitary component. In other instances, as depicted in FIGS. 18-19, the main housing 1012b can comprise one or more segments that are formed as separate components that are coupled together (e.g., via fasteners, adhesive, mating features, and/or other means for coupling). For example, the main housing 1012b can include an upper segment 1090a (FIG. 18) and a lower segment 1090b (FIG. 19) which can be coupled together.

The upper segment 1090a can include mating features 1092a connected to an inner surface 1094a of the upper segment 1090a. The lower segment 1090b can include corresponding mating features 1092b connected to an inner surface 1094b of the lower segment 1090b. The mating features 1092b of the lower segment 1090b can be configured to mate with the mating features 1092a of the upper segment 1090a. In the illustrated example, the mating features 1092a can include pins and the corresponding mating features 1092b can include sockets. It should be appreciated that other mating features are contemplated for the upper segment 1090a and the lower segment 1090b, including without limitation clips and corresponding clip tabs, and/or hooks and corresponding hinges, etc. In some instances, the mating features 1092a of the upper segment 1090a can include the sockets and the corresponding mating features 1092b of the lower segment 1090b can include the pins. In some instances, as depicted, all of the mating features on one of the segments (e.g., upper segment 1090a) can include one type of mating features (e.g., pins). In other instances, the mating features on one of the segments (e.g., 1090a or 1090b) can include a combination of different types of mating features. Additionally, in the illustrated example, six mating features 1092a, 1092b are included on each segment 1090a, 1090b, respectively. In other examples, a different number of mating features may be included (e.g., fewer than six, more than six, etc.) on each segment.

The upper segment 1090a can include grooves 1096a and internal ribs 1098a configured to receive and retain components of the indicator assembly 1017, as described in more detail below. The grooves 1096a can be included in an outer wall 1100a of the upper segment 1090a. The internal ribs 1098a can be positioned radially inward of the grooves 1096a (e.g., internal to the outer wall 1100a, etc.). The ribs 1098a can define an opening or slot and a component of the indicator assembly 1017 can be positioned within the slot defined by the ribs 1098a. In the illustrated example, the ribs 1098a can be positioned adjacent to the mating features 1092a and extend from the mating features 1092a in an axial direction.

The upper segment 1090a can include a platform 1102a (which can also be referred to herein as an “alignment platform”) extending from the inner surface 1094a. The platform 1102a can be positioned in a central region of the upper segment 1090a (e.g., halfway between ends of the upper segment 1090a in an axial direction, etc.). The platform 1102a can be configured to position the spine assembly 1014 relative to the housing 1012. Specifically, the platform 1102a can align the distal spine 1024 and the proximal spine 1026 relative to the upper segment 1090a of the main housing 1012b. In some instances, the platform 1102a can include notches to accommodate the barrels 1082 (FIG. 6).

Similar to the upper segment 1090a, the lower segment 1090b can include grooves 1096b and internal ribs 1098b configured to receive and retain components of the indicator assembly 1017. The grooves 1096b can be included in an outer wall 1100b of the lower segment 1090b. The internal ribs 1098b can be positioned radially inward of the grooves 1096b (e.g., internal to the outer wall 1100b, etc.). The ribs 1098b can define an opening or slot and a component of the indicator assembly 1017 can be positioned within the slot defined by the ribs 1098b. In the illustrated example, the ribs 1098b can be positioned adjacent to the mating features 1092b and extend from the mating features 1092b in an axial direction.

The lower segment 1090b can include a platform 1102b (which can also be referred to herein as an “alignment platform”) extending from the inner surface 1094b. The platform 1102b can be positioned in a central region of the lower segment 1090b (e.g., halfway between ends of the lower segment 1090b in an axial direction, etc.). The platform 1102b can be configured to position the spine assembly 1014 relative to the housing 1012. Specifically, the platform 1102b can align the spine assembly 1014 relative to the lower segment 1090b of the main housing 1012b. In some instances, as best shown in FIG. 6, the platform 1102b can contact the base 1062 of the distal spine 1024 to achieve this alignment. In some examples, the spine assembly 1014 can be configured such that the spine assembly 1014 can only be assembled within the housing 1012 in one direction or orientation based on mating features of the housing 1012. For example, the base 1062 of the spine assembly 1014 and the platform 1102b can be configured such that the base 1062 can only be aligned or mated with the platform 1102b in one direction (e.g., with the spine shaft 1060 extending distally from the base 1062, etc.).

The lower segment 1090b can include bores 1104 (e.g., threaded bores, etc.) configured to receive fasteners 1106 (e.g., screws, bolts, etc.). With additional reference to FIG. 20, when the spine assembly 1014 and the housing 1012 are coupled (e.g., fastened, etc.) together, the bores 1104 of the lower segment 1090b of the main housing 1012b and the holes 1058 of the distal spine 1024 and the proximal spine 1026 can align. In this manner, the fasteners 1106 can be inserted through the holes 1058 and into the bores 1104 to attach the spine assembly 1014 to the housing 1012.

Referring now to FIGS. 20-22, the indicator assembly 1017 (also referred to herein as a “flex indicator assembly”) can be positioned within the main housing 1012b. The indicator assembly 1017 can be configured to indicate the amount of flex that is being applied to the distal end portion 1005 of the delivery shaft 1004 by the adjustment mechanism 1016. The indicator assembly 1017 can comprise windows 1108 and indicators 1110 (also referred to herein as “flex indicators” or “flex measuring devices”) that can be positioned within the main housing 1012b and can be viewable through the windows 1108 (e.g., adjacent to the windows 1108, etc.).

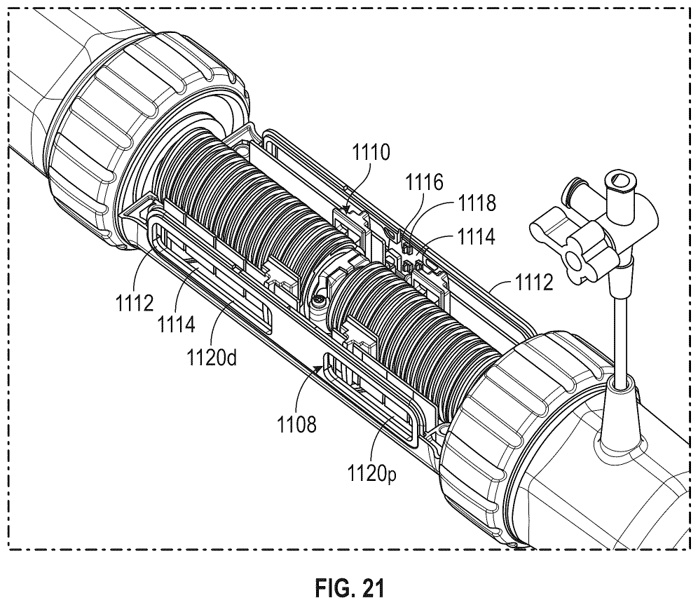

The windows 1108 can be positioned in the grooves 1096a, 1096b of the upper and lower segments 1090a, 1090b of the main housing 1012b. The windows 1108 can include frames 1112 and windowpanes 1114 that can be coupled to the frames 1112. For example, a windowpane 1114 can be retained within a frame 1112 via a snap and/or friction fit. In some instances, as depicted in FIG. 21, the frames 1112 can include retaining members 1116, such as tabs, clips, or the like, that extend radially from the frame 1112 and can engage with corresponding openings 1118 of the windowpanes 1114. The windowpanes 1114 can be transparent, translucent, or the like to enable a user to view the indicators 1110 through the windows 1108.

In the illustrated example, the indicator assembly 1017 can comprise frames 1112 disposed on opposite sides of the handle assembly 1002. Each frame 1112 can be coupled to one windowpane 1114 and can define a distal viewing region 1120d and a proximal viewing region 1120p (collectively referred to as viewing regions 1120). In the illustrated example, the retaining members 1116 of the frames 1112 and the openings 1118 of the windowpanes 1114 are positioned between the viewing regions 1120.

The indicator assembly 1017 can also comprise four indicators 1110, as shown. Two of the indicators 1110 can be disposed on each side of the handle assembly 1002. In some examples, the two distal indicators 1110 can indicate an amount of one type of curvature or flex (e.g., in a particular direction, as controlled by knob 1008a, etc.) and the two proximal indicators 1110 can indicate an amount of a different type of curvature or flex (e.g., in a different direction, as controlled by knob 1008b, etc.). The distal indicators 1110 can be viewable through the distal viewing region 1120d of the windows 1108 and the proximal indicators 1110 can be viewable through the proximal viewing region 1120p of the windows 1108.

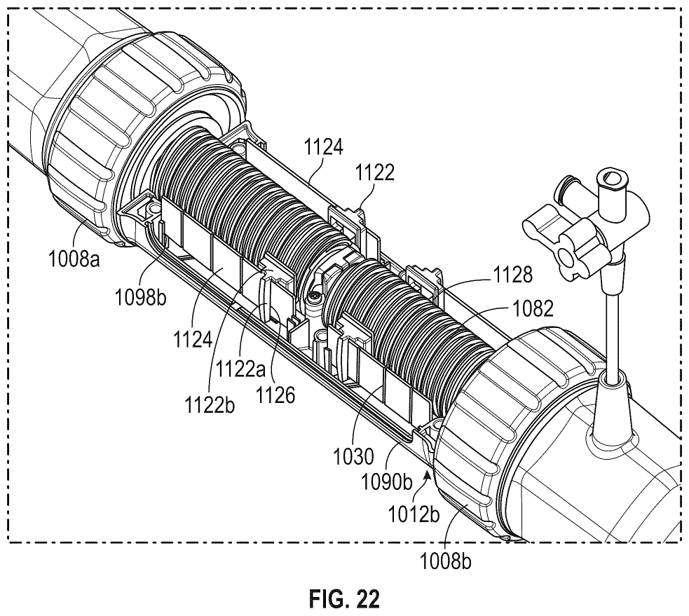

Each indicator 1110 comprises a slider 1122 and a background member 1124, as best shown in FIG. 22. The background members 1124 can be positioned within the internal ribs 1098a, 1098b of the upper and lower segments 1090a, 1090b of the main housing 1012b. In this way, the background members 1124 can be fixed relative to the housing 1012 (e.g., prevented from moving in an axial direction relative to the housing 1012, etc.). The slider 1122 can be configured to slide axially relative to the background member 1124. In particular, the slider 1122 can include an opening 1126 and the background member 1124 can pass through the opening 1126. In this way, the slider 1122 can include an outer portion 1122a that is visible or viewable through the viewing regions 1120 and an inner portion 1122b that is not visible or viewable through the viewing regions 1120. In particular, the outer portion 1122a can be positioned outward of the background member 1124 in a radial direction and the inner portion 1122b can be positioned inward of the background member 1124 in a radial direction. The outer portion 1122a can also be referred to as the “visible portion” or “viewable portion” of the slider 1122.

The slider 1122 can also include at least one projection 1128 (e.g., one or more tabs, pins, etc.) extending from the inner portion 1122b of the slider 1122. The projections 1128 can be configured to engage with a corresponding barrel 1082. In this way, as the barrel 1082 is rotated by one of the knobs (e.g., 1008a or 1008b, etc.), the threads of the barrel 1082 can cause the slider 1122 via the projection 1128 to move axially relative to the background member 1124.

In the illustrated example, the viewable portion 1122a of the slider 1122 can be configured as a bar. As such, the slider 1122 can also be referred to as a “slider bar. ” It should be appreciated that in other instances, the viewable portion 1122a of the slider 1122 can include other configurations such as, for example, tabs extending in front of only a portion of the background members 1124 or the like.

In some instances, one background member 1124 can be used for two indicators 1110. For example, in these instances, the background member 1124 can extend axially such that the extended background member 1124 is visible through both a distal viewing region 1120d and a proximal viewing region 1120p. Additionally, two sliders 1122 can be positioned on the extended background member 1124.

The background members 1124 can include indicia 1130 (e.g., markings, hash marks, etc.) to indicate an amount and/or measure of flex. For example, in some instances, as the slider 1122 moves proximally relative to the background member 1124, the position of the slider 1122 relative to the indicia 1130 can indicate an increased amount of flex. Conversely, in some instances, as the slider 1122 moves distally relative to the background member 1124, the position of the slider 1122 relative to the indicia 1130 can indicate a decreased amount of flex. It should be appreciated that movement of the slider 1122 relative to the background member 1124 in the axial direction can indicate either an increased or decreased amount of flex.

In some instances, the indicator assembly 1017 can include additional indicia (e.g., colors, markings, etc.) to distinguish the distal indicators 1110 (e.g., indicating one type of curvature or flex, etc.) from the proximal indicators 1110 (e.g., indicating a different type of curvature or flex, etc.). For example, the slider 1122, the background member 1124, and/or the indicia 1130 of the distal indicators 1110, the portion of the frames 1112 surrounding the distal viewing regions 1120d, and/or the corresponding knob 1008a can comprise a first color, marking, or the like to indicate that these components are related (e.g., to indicate that flex caused by rotation of the knob 1008a is viewable through the distal viewing region 1120d and indicated by the distal indicators 1110, etc.). Similarly, in some instances, the slider 1122, the background member 1124, and/or the indicia 1130 of the proximal indicators 1110, the portion of the frames 1112 surrounding the proximal viewing regions 1120p, and/or the corresponding knob 1008b can comprise a second color, marking, or the like to distinguish from the first. It should be appreciated that other components can include additional indicia to distinguish the different types of curvature or flex provided by the adjustment mechanism 1016 and indicated by the indicator assembly 1017.

As introduced above, the seal assembly 1018 and the locking mechanism 1020 can be positioned within the proximal housing 1012c. In some instances, the proximal housing 1012c can be integrally formed as a single, unitary component. In other instances, as depicted in FIGS. 23-25, the proximal housing 1012c can comprise one or more segments that are formed as separate components that are coupled together (e.g., via fasteners, adhesive, mating features, and/or other means for coupling). For example, the proximal housing 1012c can include two segments which are configured to be coupled together with mating features 1132 (e.g., pins, sockets, etc.). FIG. 24 depicts the delivery apparatus 1000 with one of the segments of the proximal housing 1012c removed.

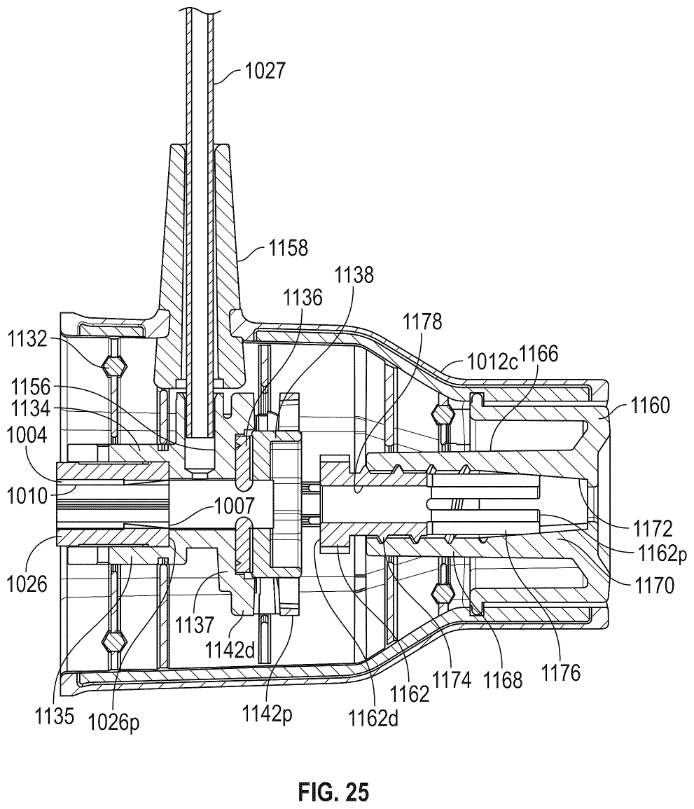

The seal assembly 1018 can be configured to surround (e.g., cover) the proximal spine 1026 and the delivery shaft 1004, as shown in FIG. 25. Specifically, the seal assembly 1018 can be positioned around the proximal end 1026p of the proximal spine 1026 and a proximal end 1007 of the delivery shaft 1004. The seal assembly 1018 can include a seal housing 1134, a seal 1136, and a seal compressor member 1138.

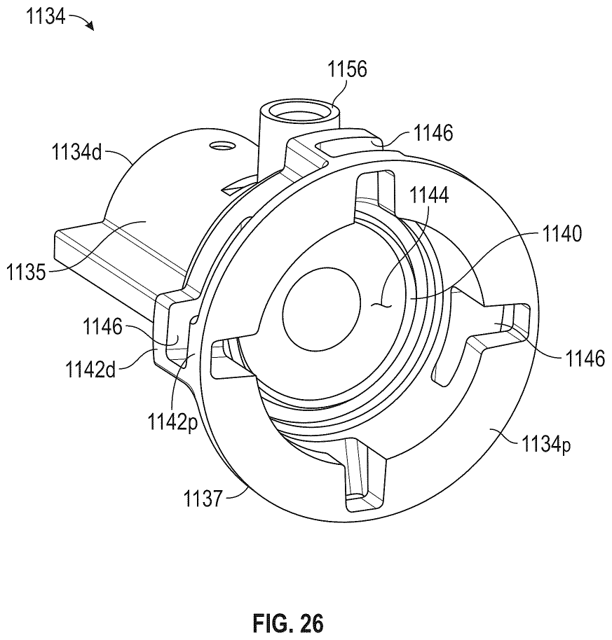

The seal housing 1134 can include a shaft portion 1135 and a head portion 1137. The shaft portion 1135 can be positioned at a distal end 1134d of the seal housing 1134 and the head portion 1137 can be positioned at a proximal end 1134p of the seal housing 1134. Additionally, the seal 1136 can be positioned within the seal housing 1134. In particular, as best shown in FIG. 26, the head portion 1137 of the seal housing 1134 can include a seat 1140 for the seal 1136. The seat 1140 can be defined by an inner surface of the head portion 1137 of the seal housing 1134. The seat 1140 can be configured to prevent the seal 1136 from moving relative to the seal housing 1134.

The head portion 1137 can include a distal flange 1142d and a proximal flange 1142p. The proximal flange 1142p can be positioned at the proximal end 1134p of the seal housing 1134. The proximal flange 1142p can include an opening 1144 which has a shape corresponding to the seal compressor member 1138. In this manner, the seal compressor member 1138 can be inserted into the head portion 1137 of the seal housing 1134 through the opening 1144. The seal housing 1134 can also include locking channels 1146 positioned in the head portion 1137 and in fluid communication with the opening 1144. The locking channels 1146 can be partially defined by the flanges 1142d, 1142p. In the illustrated example, the seal housing 1134 includes four locking channels 1146, although the seal housing 1134 can include a different number of locking channels 1146 in other examples. The locking channels 1146 can be configured to receive and releasably retain the seal compressor member 1138 after the seal compressor member 1138 is inserted into the seal housing 1134.

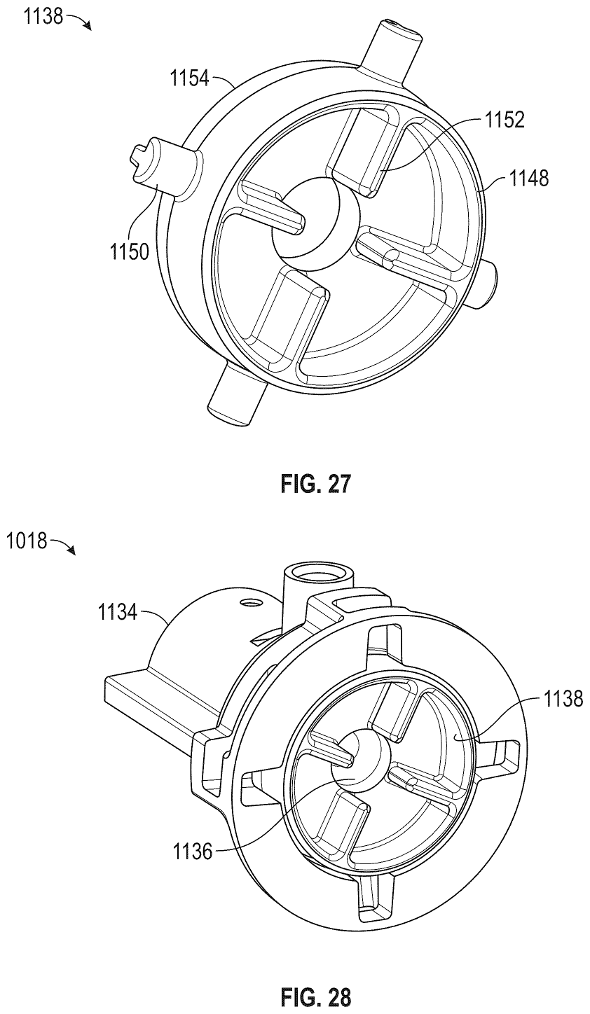

As shown in FIG. 27, the seal compressor member 1138 can include a base 1148 and pins 1150 extending outwardly from the base 1148 in a radial direction. In the illustrated example, the seal compressor member 1138 includes four pins 1150 corresponding to the four locking channels 1146 of the seal housing 1134. However, it should be appreciated that in other examples, the seal compressor member 1138 can include a different number of pins 1150 (e.g., more or less than four, etc.). The seal compressor member 1138 can also include one or more ribs 1152. In some instances, the ribs 1152 can be configured to be gripped by a user (e.g., to rotate the seal compressor member 1138 about the central longitudinal axis 1003, etc.).

The seal assembly 1018 can be configured to axially compress the seal 1136 within the seal housing 1134 (e.g., without use of screws, etc.). In particular, the seal compressor member 1138 can be inserted into the opening 1144 at the proximal end 1134p of the seal housing 1134 and can be positioned adjacent to the seal 1136. Specifically, a distal surface 1154 of the seal compressor member 1138 can contact the seal 1136. A force can be applied to the seal compressor member 1138 in an axial direction to axially compress the seal 1136. In this manner, the seal 1136 is compressed between the seat 1140 of the seal housing 1134 and the distal surface 1154 of the seal compressor member 1138. When compressed, the seal 1136 can be configured to slightly pinch or squeeze a shaft of a delivery device (e.g., any of the pusher assemblies described herein, etc.) inserted through the main lumen 1010 of the delivery apparatus 1000 in a radial direction (FIG. 25).

When the seal 1136 is compressed by the seal compressor member 1138, the pins 1150 of the seal compressor member 1138 can be distal to the flange 1142p and positioned within the locking channels 1146. In this position, the seal compressor member 1138 can be rotated relative to the seal housing 1134 (e.g., about the axis 1003, etc.) to lock the seal compressor member 1138 into place, as shown in FIG. 28. In particular, the pins 1150 of the seal compressor member 1138 can be guided through the locking channels 1146 to a locked position. In the locked position, the pins 1150 can be in contact with a surface of the flange 1142p which enables the force to be maintained on the seal 1136. This results in a homeostatic pressure being applied to the seal 1136 in an axial direction.

In some instances, as depicted, the seal housing 1134 can include a flush port 1156. As depicted, the flush port 1156 can be distal to the seal 1136 and extend radially from the shaft portion 1135. The flush tube 1027 can be coupled to the flush port 1156 (e.g., to an inner surface of the flush port 1156, etc.). In some instances, the flush tube 1027 can comprise a flexible or semi-flexible material and the proximal housing 1012c can comprise a rigid material. To reinforce the flush tube 1027 at the location where the flush tube 1027 passes through the proximal housing 1012c, a support 1158 can be positioned around the flush tube 1027 and coupled to the proximal housing 1012c. The support 1158 can comprise a flexible or semi-flexible material (e.g., rubber, etc.).

FIG. 29 illustrates the delivery apparatus 1000 with a pusher assembly 1180 (e.g., similar to pusher assembly 58, etc.) extending proximally from the delivery apparatus 1000.

Specifically, the pusher assembly 1180 can be positioned within the main lumen 1010 of the delivery apparatus 1000 (e.g., within the delivery shaft 1004, etc.). In some examples, the delivery apparatus 1000 can comprise the pusher assembly 1180, such that the delivery apparatus 1000 comprises the handle assembly 1002, the delivery shaft 1004 and the pusher assembly 1180. As described above, the pusher assembly 1180 can be used to deploy and/or implant a docking device at an implantation site. To do so, it can be necessary for the pusher assembly 1180 to move or slide relative to the delivery apparatus 1000 in an axial direction in some instances. It can also be necessary for the pusher assembly 1180 to remain stationary relative to the delivery apparatus 1000 in other instances.

The locking mechanism 1020 can be configured to prevent movement of the pusher assembly 1180 relative to the delivery apparatus 1000 when the locking mechanism 1020 is in the locked configuration. In the unlocked configuration, the locking mechanism 1020 can be configured to allow such movement. Referring again to FIGS. 23-25, the locking mechanism 1020 can include a rotatable knob 1160 (also referred to herein as a “locker body”) and a collet 1162. The knob 1160 includes two tabs 1164 extending outwardly from the knob 1160 in a radial direction and a shaft 1166 extending distally from the knob 1160 in an axial direction. The shaft 1166 can be configured to receive the collet 1162. In the illustrated example, the shaft 1166 can include a threaded region 1168 having internal threads and a tapered region 1170. The tapered region 1170 includes an inner surface 1172 that can be tapered from a larger inner diameter to a smaller inner diameter in a proximal direction.

The collet 1162 can include external threads 1174 configured to engage with the internal threads in the threaded region 1168 of the shaft 1166. The collet 1162 can also include axially extending projections 1176 (also referred to herein as “cantilevered arms”) at a proximal end 1162p of the collet 1162. In some instances, as depicted, the collet 1162 can include four projections 1176. It should be appreciated that in other instances, the collet 1162 can include a different number of projections 1176. The collet 1162 can also include a central lumen 1178 extending from a distal end 1162d to the proximal end 1162p of the collet 1162. The central lumen 1178 can be coaxial with the main lumen 1010 of the delivery apparatus 1000.

When the locking mechanism 1020 is in the unlocked configuration, as shown in FIG. 25, the projections 1176 extend straight out from the collet 1162. In other words, the diameter of the central lumen 1178 is uniform from the distal end 1162d to the proximal end 1162p of the collet 1162. In the unlocked configuration, the diameter of the central lumen 1178 can be the same as the diameter of the main lumen 1010. Rotating the knob 1160 by a certain amount (e.g., a quarter turn, etc.) with respect to the central longitudinal axis 1003 can change the locking mechanism 1020 from the unlocked configuration to the locked configuration. As such, in some instances, the locking mechanism 1020 can also be referred to as a “quarter turn locking mechanism.”As the knob 1160 is rotated, the collet 1162 can move in an axial direction relative to the knob 1160 towards the tapered region 1170 of the shaft 1166. When the locking mechanism 1020 is in the locked configuration, the projections 1176 can contact the inner surface 1172 of the shaft 1166 and can be pushed or flexed radially inwards by the taper of the inner surface 1172. In other words, in the locked configuration, the diameter of the central lumen 1178 is smaller at the proximal end 1162p of the collet 1162 than at the distal end 1162d of the collet 1162 due to the tapered inner surface 1172. In this manner, the projections 1176 can be configured to clamp around a device inserted through the delivery apparatus 1000 (e.g., pusher assembly 1180, etc.) to lock the device in place. As such, the projections 1176 can be configured to prevent movement of the device relative to the delivery apparatus 1000 (e.g., relative to the main lumen 1010, the handle 1006, etc.).

Further details of the docking device delivery apparatus and its variants are described in International Patent Application No. PCT/US2021/052669, which is incorporated by reference herein in its entirety.

Referring again to FIG. 23, in some instances, as depicted, the proximal housing 1012c can include proximal extensions 1182 (e.g., axial extensions or projections, etc.) that can extend over the knob 1160 in an axial direction. The extensions 1182 can be configured to prevent the knob 1160 from being rotated more than a certain angular amount (e.g., more than a quarter turn, etc.). In particular, the extensions 1182 can project axially over the knob 1160 between the tabs 1164. Additionally, the tabs 1164 can extend outwards in a radial direction farther than the extensions 1182. In this way, the tabs 1164 can contact the extensions 1182 to prevent additional rotation. The extensions 1182 can be sized and/or spaced such that a full rotation of the knob 1160 between the extensions 1182 (e.g., a quarter turn, etc.) can transition the locking mechanism 1020 from the unlocked configuration to the locked configuration (and vice versa).

FIG. 30 illustrates an example delivery system 2000 including the delivery apparatus 1000, the pusher assembly 1180, a guide catheter 2002 (e.g., similar to guide catheter 30, etc.), and a stabilizer 2004 (also referred to herein as a “stabilizing tower”or “stabilizing device”).

The delivery system 2000 can be used in a transcatheter heart valve replacement procedure, for example, as described above with reference to FIGS. 1-4. Specifically, the delivery system 2000 depicted in FIG. 30 can be used during the second stage of the procedure described above with reference to FIG. 2A. As shown in FIG. 30, the delivery apparatus 1000 and the pusher assembly 1180 can be inserted through the guide catheter 2002. Specifically, the delivery shaft 1004 of the delivery apparatus 1000 can be advanced through the guide catheter 2002 (e.g., through a central lumen thereof, etc.). A shaft of the pusher assembly 1180 can be inserted through the delivery apparatus 1000 and extend distally through the delivery apparatus 1000 into the delivery shaft 1004. As described above, the locking mechanism 1020 can be configured to selectively allow movement (e.g., axial and/or rotational movement, etc.) of the pusher assembly 1180 relative to the delivery apparatus 1000.

The guide catheter 2002 and the delivery apparatus 1000 can be coupled to the stabilizer 2004 which can support and stabilize the guide catheter 2002 and the delivery apparatus 1000 (e.g., during the procedure, etc.). The stabilizer 2004 can include supports 2006 (e.g., clips, clamps, braces, etc.) which can be configured to hold or grip the guide catheter 2002 and the delivery apparatus 1000. In some instances, the supports 2006 can be slidably coupled to the stabilizer 2004 and can be relocated or repositioned on the stabilizer 2004 in an axial direction. In some examples, as depicted, a support 2006 can be configured to engage with the cap 1046 of the delivery apparatus 1000. For example, as described above, the cap 1046 can be fixed relative to the rest of the delivery apparatus 1000 (e.g., the spine extension 1022 and the nose cone 1012a, etc.). In some examples, torque may be exerted on the handle assembly 1002 of the delivery apparatus 1000 while the delivery apparatus 1000 is coupled to the stabilizer 2004. This torque can be transferred to the delivery shaft 1004 via the spine assembly 1014, as described above.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A handle for a delivery apparatus, the handle comprising: a housing having an outer wall; at least one window coupled to the outer wall of the housing, the at least one window defining at least one viewing region through the outer wall; and at least one indicator positioned within the housing adjacent to the window, the at least one indicator including a background member and a slider, wherein the slider is configured to slide relative to the background member.

Example 2. The handle of any example herein, particularly example 1, wherein the slider includes an opening and the background member extends through the opening of the slider.

Example 3. The handle of any example herein, particularly either example 1 or example 2, wherein the slider includes a viewable portion that is positioned between the background member and the window.

Example 4. The handle of any example herein, particularly example 3, wherein the viewable portion of the slider is a bar.

Example 5. The handle of any example herein, particularly any one of examples 1-4, wherein the background member is fixed relative to the housing.

Example 6. The handle of any example herein, particularly any one of examples 1-5, wherein the housing further comprises ribs extending from an inner surface of the housing, wherein the ribs define a slot configured to receive the background member.

Example 7. The handle of any example herein, particularly any one of examples 1-6, wherein the at least one window includes a frame and a windowpane coupled to the frame.

Example 8. The handle of any example herein, particularly example 7, wherein the frame defines two viewing regions, wherein each viewing region is associated with a different measurement indicated by the at least one indicator.

Example 9. The handle of any example herein, particularly example 7, wherein the frame includes retaining members extending from an inner surface of the frame, wherein the retaining members couple the windowpane to the frame, and wherein the retaining members are positioned between the two viewing regions.

Example 10. The handle of any example herein, particularly any one of examples 1-9, wherein a first window is positioned on a first side of the handle, and wherein a second window is positioned on a second, opposite side of the handle.

Example 11. The handle of any example herein, particularly example 10, wherein the at least one indicator includes four indicators, wherein two indicators are positioned adjacent the first window and wherein the other two indicators are positioned adjacent the second window.

Example 12. The handle of any example herein, particularly example 11, wherein the two indicators include two sliders and only one background member.

Example 13. The handle of any example herein, particularly example 11, wherein the two indicators include two sliders and two background members.

Example 14. The handle of any example herein, particularly any one of examples 1-13, further comprising an adjustment mechanism operatively connected to the slider, wherein the slider is configured to indicate an amount of adjustment by the adjustment mechanism.

Example 15. A delivery apparatus comprising: a handle including a body and a nose cone positioned distal to the body, wherein the nose cone includes at least one axial runner extending from an inner surface of the nose cone; a spine positioned within the body of the handle, the spine including a lumen; a shaft positioned within the lumen of the spine, the shaft extending distally from the handle; and a spine extension positioned within the nose cone and coupled to the spine, the spine extension at least partially surrounding the shaft, the spine extension including radial projections, wherein each of the radial projections includes one or more mating features defining at least one slot corresponding to the at least one axial runner of the nose cone.

Example 16. The delivery apparatus of any example herein, particularly example 15, wherein the spine extension partially surrounds the shaft in a circumferential direction, wherein the spine extension is configured to transmit torque to the shaft.

Example 17. The delivery apparatus of any example herein, particularly either example 15 or example 16, wherein the nose cone includes a distal end portion extending partially around the shaft in a circumferential direction, wherein the spine extension includes a distal end portion extending partially around the shaft in the circumferential direction, and wherein the distal end portions of the nose cone and the spine extension fully surround the shaft in the circumferential direction.

Example 18. The delivery apparatus of any example herein, particularly example 17, further comprising a cap disposed around the distal end portions of the nose cone and the spine extension and configured to limit movement of the nose cone relative to the spine extension.

Example 19. The delivery apparatus of any example herein, particularly example 18, wherein the cap is configured for attachment to an external stabilizing device.

Example 20. The delivery apparatus of any example herein, particularly either example 18 or example 19, wherein the cap comprises an elastomeric material.

Example 21. The delivery apparatus of any example herein, particularly any one of examples 18-20, wherein the cap includes an inner surface defining a hexagonal shape, and wherein the distal end portions of the nose cone and the spine extension define a complementary hexagonal shape.

Example 22. The delivery apparatus of any example herein, particularly any one of examples 15-21, wherein the radial projections are spaced apart from each other in an axial direction and/or a circumferential direction.

Example 23. The delivery apparatus of any example herein, particularly any one of examples 15-22, wherein a mating feature of a first radial projection and a mating feature of a second, axially-adjacent radial projection define a slot corresponding to an axial runner.

Example 24. The delivery apparatus of any example herein, particularly any one of examples 15-23, wherein the axial runner includes one or more panels and a tip, wherein the tip is configured to mate with the at least one slot defined by the mating features of the radial projections.

Example 25. The delivery apparatus of any example herein, particularly example 24, wherein the axial runner includes a primary panel including the tip and at least one secondary panel angled relative to the primary panel.

Example 26. The delivery apparatus of any example herein, particularly any one of examples 15-25, wherein a proximal end of the spine extension is positioned within the body.

Example 27. The delivery apparatus of any example herein, particularly any one of examples 15-26, wherein a distal end of the spine is positioned within the nose cone.

Example 28. The delivery apparatus of any example herein, particularly any one of examples 15-27, wherein the spine includes a distal spine and a proximal spine, wherein the distal spine is coupled to the proximal spine via mating features.

Example 29. The delivery apparatus of any example herein, particularly example 28, wherein one of the distal spine and the proximal spine includes a base, wherein the base contacts an inner surface of the body.

Example 30. The delivery apparatus of any example herein, particularly example 29, wherein the base is the only portion of the spine that directly contacts the inner surface of the body.

Example 31. The delivery apparatus of any example herein, particularly any one of examples 15-30, wherein the shaft includes a first portion having a first outer diameter and a second portion having a second, smaller outer diameter.

Example 32. The delivery apparatus of any example herein, particularly example 31, wherein the spine extension partially surrounds the first portion of the shaft and the second portion of the shaft.

Example 33. The delivery apparatus of any example herein, particularly either example 31 or example 32, wherein the second portion of the shaft includes one or more openings.

Example 34. A delivery apparatus comprising: a handle; a shaft having a distal end and a proximal end, the shaft extending distally from the handle, wherein the proximal end of the shaft is positioned in the handle, the shaft including a lumen extending from the distal end to the proximal end; and a seal assembly positioned in the handle and coupled to the proximal end of the shaft, the seal assembly including a seal housing, a seal disposed within the seal housing, and a seal compressor member coupled to the seal housing and configured to compress the seal within the seal housing in an axial direction.

Example 35. The delivery apparatus of any example herein, particularly example 34, wherein the seal compressor member includes a base and pins extending radially from the base.

Example 36. The delivery apparatus of any example herein, particularly either example 34 or example 35, wherein the seal housing includes a head portion and a shaft portion, wherein the seal is positioned within the head portion, and wherein the shaft is positioned within the shaft portion.

Example 37. The delivery apparatus of any example herein, particularly example 36, wherein a proximal end of the head portion includes an opening configured to receive the seal compressor member.

Example 38. The delivery apparatus of any example herein, particularly either example 36 or example 37, wherein the head portion includes one or more flanges defining locking channels for the seal compressor member, the locking channels configured to maintain compression of the seal by the seal compressor member.

Example 39. The delivery apparatus of any example herein, particularly example 38, wherein the pins of the seal compressor member are distal to a flange at the proximal end of the head portion.

Example 40. The delivery apparatus of any example herein, particularly either example 38 or example 39, wherein the locking channels are in fluid communication with the opening and extend in a circumferential direction.

Example 41. The delivery apparatus of any example herein, particularly any one of examples 34-40, wherein the handle comprises the handle of any one of examples 1-14.

Example 42. A delivery apparatus comprising: a handle having at least one window; a delivery shaft extending distally from the handle, the delivery shaft having a distal end and a proximal end, wherein the proximal end of the delivery shaft is positioned within the handle; a spine assembly positioned at least partially around the delivery shaft, the spine assembly coupled to the handle; an adjustment mechanism configured to adjust a curvature of the distal end of the delivery shaft; and an indicator configured to indicate an amount of adjustment by the adjustment mechanism, the indicator positioned within the handle and viewable through the at least one window.

Example 43. The delivery apparatus of any example herein, particularly example 42, wherein the handle includes a housing having a distal segment, an intermediate segment, and a proximal segment.

Example 44. The delivery apparatus of any example herein, particularly example 43, wherein the spine assembly includes a spine extension positioned at least partially around the delivery shaft, the spine extension having radial projections.

Example 45. The delivery apparatus of any example herein, particularly example 44, wherein the distal segment of the housing includes mating elements corresponding to the radial projections.

Example 46. The delivery apparatus of any example herein, particularly example 43, wherein the at least one window is disposed in the intermediate segment of the housing.

Example 47. The delivery apparatus of any example herein, particularly any one of examples 43-46, wherein the indicator includes a slider operatively connected to the adjustment mechanism and a background member positioned between the at least one window and the adjustment mechanism.

Example 48. The delivery apparatus of any example herein, particularly example 47, wherein the slider includes an opening, wherein the background member is positioned within the opening, wherein the slider is configured to move axially relative to the background member.

Example 49. The delivery apparatus of any example herein, particularly either example 47 or example 48, wherein the background member is coupled to the intermediate segment of the housing.

Example 50. The delivery apparatus of any example herein, particularly any one of examples 47-49, wherein the background member includes indicia.

Example 51. The delivery apparatus of any example herein, particularly any one of examples 42-50, further comprising a seal assembly coupled to the delivery shaft, the seal assembly including a seal housing, a seal positioned within the seal housing, and a seal compressor member configured to axially compress the seal within the seal housing.

Example 52. The delivery apparatus of any example herein, particularly any one of examples 42-51, further comprising a locking mechanism at a proximal end of the handle, the locking mechanism including a rotatable knob and a collet.

Example 53. The delivery apparatus of any example herein, particularly example 52, wherein rotation of the knob is configured to transition the locking mechanism between an unlocked configuration and a locked configuration.

Example 54. The delivery apparatus of any example herein, particularly either example 52 or example 53, wherein the knob includes radial tabs and wherein the proximal segment of the housing includes axial extensions configured to engage with the radial tabs to limit rotational movement of the knob relative to the housing.

Example 55. The delivery apparatus of any example herein, particularly any one of examples 52-54, wherein the knob includes a lumen having a threaded region and a tapered region, wherein the collet is moveable within the lumen in an axial direction relative to the knob.

Example 56. The delivery apparatus of any example herein, particularly any one of examples 52-55, further comprising a pusher assembly including a shaft, wherein the shaft is positioned within a lumen of the delivery shaft.

Example 57. The delivery apparatus of any example herein, particularly example 56, wherein the locking mechanism is configured to prevent movement of the shaft of the pusher assembly relative to the handle in the locked configuration.

Example 58. An assembly comprising: a delivery apparatus including the handle of any one of examples 1-14; and a prosthetic implant releasably coupled to the delivery apparatus.

Example 59. The assembly of any example herein, particularly example 58, wherein the prosthetic implant comprises a docking device.

Example 60. The assembly of any example herein, particularly example 59, wherein the docking device includes a coil.

Example 61. The assembly of any example herein, particularly example 58, wherein the prosthetic implant comprises a prosthetic valve.

Example 62. The assembly of any example herein, particularly any one of examples 58-61, wherein the delivery apparatus comprises the delivery apparatus of any one of examples 15-57.

Example 63. The assembly of any example herein, particularly any one of examples 58-62, further comprising a guide catheter, wherein at least a portion of the delivery apparatus extends through a lumen of the guide catheter.

Example 64. The assembly of any example herein, particularly any one of examples 58-63, further comprising a stabilizer coupled to at least the delivery apparatus.

Example 65. The assembly of any example herein, particularly any one of examples 58-64, further comprising a pusher assembly, wherein at least a portion of the pusher assembly extends through a lumen of the delivery apparatus.

Example 66. An assembly comprising: the delivery apparatus of any one of examples 15-57; and a prosthetic implant releasably coupled to the delivery apparatus.

Example 67. The assembly of any example herein, particularly example 66, wherein the prosthetic implant comprises a docking device.

Example 68. The assembly of any example herein, particularly example 67, wherein the docking device includes a coil.

Example 69. The assembly of any example herein, particularly example 66, wherein the prosthetic implant comprises a prosthetic valve.

Example 70. The assembly of any example herein, particularly any one of examples 66-69, further comprising a guide catheter, wherein at least a portion of the delivery apparatus extends through a lumen of the guide catheter.

Example 71. The assembly of any example herein, particularly any one of examples 66-70, further comprising a stabilizer coupled to at least the delivery apparatus.

Example 72. The assembly of any example herein, particularly any one of examples 66-71, further comprising a pusher assembly, wherein at least a portion of the pusher assembly extends through a lumen of the delivery apparatus.

Example 73. A method for implanting a prosthetic implant, the method comprising: inserting a delivery apparatus into vasculature of a patient, wherein the delivery apparatus comprises the delivery apparatus of any one of examples 15-57; and causing a first slider to slide relative to a first background member within a handle of the delivery apparatus, wherein the slider indicates an amount of a first type of adjustment of a shaft of the delivery apparatus.

Example 74. The method of any example herein, particularly example 73, wherein the delivery apparatus include a handle, wherein the handle comprises the handle of any one of examples 1-14.

Example 75. The method of any example herein, particularly either example 73 or example 74, wherein causing the first slider to slide comprises rotating a first knob of the delivery apparatus.

Example 76. The method of any example herein, particularly any one of examples 73-75, further comprising causing a second slider to slide relative to the first background member, wherein the second slider indicates an amount of a second type of adjustment of the shaft of the delivery apparatus.

Example 77. The method of any example herein, particularly any one of examples 73-75, further comprising causing a second slider to slide relative to a second background member, wherein the second slider indicates an amount of a second type of adjustment of the shaft of the delivery apparatus.

Example 78. The method of any example herein, particularly either example 76 or example 77, wherein causing the second slider to slide comprises rotating a second knob of the delivery apparatus.

Example 78. A method for implanting a prosthetic implant, the method comprising: inserting a shaft of a delivery apparatus into vasculature of a patient, wherein the delivery apparatus includes a handle external to the patient, wherein the handle comprises the handle of any one of examples 1-14; and rotating the delivery apparatus relative to the vasculature of the patient, wherein the handle comprises a support member configured to transmit torque from the handle to a distal end of the shaft.

Example 79. The method of any example herein, particularly example 78, wherein the delivery apparatus comprises the delivery apparatus of any one of examples 15-57.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A handle for a prosthetic implant delivery apparatus, the handle comprising:
a first knob configured for controlling a first mechanism of the prosthetic implant delivery apparatus, wherein the first knob comprises a first color; and
a second knob configured for controlling a second mechanism of the prosthetic implant delivery apparatus, wherein the second knob comprises a second color, and wherein the second color is visually distinguishable from the first color.

2. The handle of claim 1, wherein the first knob is configured to control flex at a first region of a delivery shaft operably coupled to the handle, and wherein the second knob is configured to control flex at a second region of the delivery shaft.

3. The handle of claim 1, further comprising an adjustment mechanism, wherein the adjustment mechanism comprises a first pull wire operably coupled to the first knob and a second pull wire operably coupled to the second knob.

4. The handle of claim 1, further comprising a first indicator and a second indicator, wherein the first indicator comprises the first color is viewable through a first window of the handle and operably coupled to the first knob, and wherein the second indicator comprises the second color is viewable through a second window of the handle and operably coupled to the second knob.

5. The handle of claim 1, wherein the first knob is distal to the second knob, and wherein the first color is a lighter color than the second color.

6. The handle of claim 1, wherein the first knob and the second knob are rotatable about a common axis.

7. The handle of claim 1, wherein the first knob and the second knob comprise the same shape and include a textured grip portion.

8. A handle for a prosthetic implant delivery apparatus, the handle comprising:
a first knob operably coupled to a first mechanism and configured to control a first aspect of the prosthetic implant delivery apparatus, wherein the first knob comprises a first color; and
a first indicator viewable through a first window of the handle, the first indicator operably coupled to the first knob and configured to indicate an amount of adjustment of the first mechanism, wherein the first indicator comprises the first color.

9. The handle of claim 8, further comprising:
a second knob operably coupled to a second mechanism and configured to control a second aspect of the prosthetic implant delivery apparatus, wherein the second knob comprises a second color; and
a second indicator viewable through a second window of the handle, the second indicator operably coupled to the second knob and configured to indicate an amount of adjustment of the second mechanism, wherein the second indicator comprises the second color.

10. The handle of claim 9, wherein the first mechanism and the second mechanism control the same type of function of the prosthetic implant delivery apparatus.

11. The handle of claim 10, wherein both the first mechanism and the second mechanism control flex of a shaft coupled to the handle, wherein the first mechanism is configured to control flex of a first region of the shaft, and wherein the second mechanism is configured to control flex of a second region of the shaft.

12. The handle of claim 10, wherein the first knob is distal to the second knob.

13. The handle of claim 8, wherein the first indicator comprises a background member and a slider configured to slide relative to the background member.

14. The handle of claim 13, wherein the background member includes indicia corresponding to an amount of curvature of a distal region of a delivery shaft coupled to the handle.

15. The handle of claim 8, wherein rotation of the first knob causes the first indicator to move axially relative to a housing of the handle.

16. A method for delivering a prosthetic implant, the method comprising:
inserting a shaft of a delivery apparatus into a patient's body, wherein the shaft of the delivery apparatus is coupled to a handle disposed outside of the patient's body, wherein the handle comprises a knob, an indicator, and an adjustment mechanism, wherein the knob is operably coupled to the indicator and the adjustment mechanism, wherein the indicator is viewable on the handle, wherein the adjustment mechanism is configured to adjust an aspect of the delivery apparatus or the prosthetic implant, and wherein the knob and the indicator have matching colors indicating a common function;
manipulating the knob to adjust the aspect of the delivery apparatus or the prosthetic implant; and
observing movement of the indicator to verify the aspect of the delivery apparatus or the prosthetic implant.

17. The method of claim 16, wherein observing the indicator comprises viewing movement of a slider relative to a background member positioned within the handle.

18. The method of claim 16, further comprising deploying the prosthetic implant at an implantation location after manipulating the knob.

19. The method of claim 16, wherein the knob is a first knob, the indicator is a first indicator, and the adjustment mechanism is a first adjustment mechanism, and wherein the method further comprises:
manipulating a second knob of the handle, wherein the second knob is operably coupled to a second indicator and a second adjustment mechanism, wherein the second indicator is viewable on the handle, wherein the second adjustment mechanism is configured to adjust another aspect of the delivery apparatus or the prosthetic implant, and wherein the second knob and the second indicator have matching colors indicating another common function; and
observing movement of the second indicator to verify the another aspect of the delivery apparatus or the prosthetic implant.

20. The method of claim 19, further comprising deploying the prosthetic implant at an implantation location after manipulating the first knob and manipulating the second knob.
